(19) United States
(12) Patent Application Publication
(10) Pub. No.: US 2009/0018156 A1
(43) Pub. Date: Jan. 15, 2009

(54) Pyrrolo [2,3,B] Pyridine Derivatives Useful As RAF Kinase Inhibitors

(76) Inventors: Jun Tang, Durham, NC (US); Masato Nakano, Tokyo (JP); Toshihiro Hamajima, Tokyo (JP)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/162,811

(22) PCT Filed: Jan. 31, 2007
(86) PCT No.: PCT/US07/61351

§ 371 Date: Jul. 31, 2008

Related U.S. Application Data
(60) Provisional application No. 60/764,099, filed on Feb. 1, 2006.

(51) Int. Cl. A61K 31/437; C07D 471/04; A61P 35/00

(52) U.S. Cl. 514/300; 546/113

(57) ABSTRACT

The present invention provides pyrrolo pyridine compounds, compositions containing the same, as well as processes for the preparation and their use as pharmaceutical agents.

### FIELD OF THE INVENTION

This invention relates to novel compounds and their use as pharmaceuticals particularly as Raf kinase inhibitors for the treatment of neurotraumatic diseases, cancer, chronic neurodegeneration, pain, migraine and cardiac hypertrophy.

### BACKGROUND OF THE INVENTION

Raf protein kinases are key components of signal transduction pathways by which specific extracellular stimuli elicit precise cellular responses in mammalian cells. Activated cell surface receptors activate ras/rap proteins at the inner aspect of the plasmamembrane which in turn recruit and activate Raf proteins. Activated Raf proteins phosphorylate and activate the intracellular protein kinases MEK1 and MEK2. In turn, activated MEKs catalyse phosphorylation and activation of p42/p44 mitogen-activated protein kinase (MAPK). A variety of cytoplasmic and nuclear substrates of activated MAPK are known which directly or indirectly contribute to the cellular response to environmental change. Three distinct genes have been identified in mammals that encode Raf proteins; A-Raf, B-Raf and C-Raf (also known as Raf-1) and isoformic variants that result from differential splicing of mRNA are known.

Inhibitors of Raf kinases have been suggested for use in disruption of tumor cell growth and hence in the treatment of cancers, e.g. histiocytic lymphoma, lung adenocarcinoma, small cell lung cancer and pancreatic and breast carcinoma; and also in the treatment and/or prophylaxis of disorders associated with neuronal degeneration resulting from ischemic events, including cerebral ischemia after cardiac arrest, stroke and multi-infarct dementia and also after cerebral ischemic events such as those resulting from head injury, surgery and/or during childbirth.

### SUMMARY OF THE INVENTION

We have now found a group of novel alkyl pyrazoles that are inhibitors of Raf kinases, in particular inhibitors of B-Raf kinase.

The present invention provides a compound of formula (I):

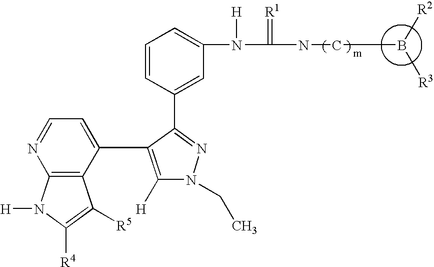

or a pharmaceutically acceptable salt or solvate thereof wherein:
R¹ is selected from O and S;
m is 0 or 1;
B is a 6 membered cycloalkyl or aryl ring;
R² and R³ are independently selected from H, alkoxy, haloalkyl, halo, and phenalkoxy;
R⁴ is selected from H, —C(O)OH, and —C(O)—O—CH₂—CH₃; and
R⁵ is selected from H and halo.

According to an embodiment, a compound of formula I is provided as described in any one of the examples.

According to another embodiment, the invention provides a compound of Formula I, a salt, or a solvate, thereof for use as an active therapeutic substance.

According to another embodiment, the invention provides a compound of Formula I, a salt, or a solvate thereof for use in the treatment of a condition mediated by inappropriate activity of at least one B-Raf family kinase.

According to another embodiment, the invention provides a pharmaceutical composition comprising compound of Formula I, a salt, or a solvate thereof and one or more pharmaceutically acceptable carriers, diluents, and excipients.

According to another embodiment, the invention provides the use of a compound of formula I, or a salt, or a solvate thereof in the manufacture of a medicament for use in the treatment of a condition mediated by inappropriate activity of at least one B-Raf family kinase.

According to another embodiment, the invention provides a method of treatment of a condition mediated by inappropriate activity of at least one B-Raf family kinase in a mammal in need thereof, with a compound of Formula I, or a salt, or a solvate thereof.

According to another embodiment, the present invention provides a method for treating a susceptible neoplasm in a mammal in need thereof, comprising: administering to the mammal, a therapeutically effective amount of a compound of formula (I) or a pharmaceutically acceptable salt or solvate thereof. Susceptible neoplasms include breast cancer, colon cancer, non-small cell lung cancer, prostate cancer, bladder cancer, ovarian cancer, gastric cancer, pancreatic cancer, carcinoma of the head and neck, esophageal carcinoma, melanoma and renal carcinoma.

According to another embodiment, the present invention provides a method for treating a susceptible neurotraumatic disease in a mammal in need thereof, comprising: administering to the mammal, a therapeutically effective amount of a compound of formula (I) or a pharmaceutically acceptable salt or solvate thereof. Susceptible neurotraumatic diseases include both open or penetrating head trauma, such as caused by surgery, or a closed head trauma injury, such as caused by an injury to the head region.

### DETAILED DESCRIPTION OF THE INVENTION

As used herein the term “raf family kinase” refers to those raf kinases and includes within its scope B-Raf.

As used herein, the terms “alkyl” (and “alkylene”) refer to straight or branched hydrocarbon chains containing from 1 to 8 carbon atoms, unless a different number of atoms is specified. Examples of “alkyl” as used herein include, but are not limited to, methyl, ethyl, n-propyl, n-butyl, n-pentyl, isobutyl, isopropyl, and tert-butyl. Examples of “alkylene” as used herein include, but are not limited to, methylene, ethylene, propylene, butylene, and isobutylene. “Alkyl” also includes substituted alkyl. The alkyl (and alkylene) groups may be optionally substituted one or more times with a halogen or hydroxyl. Thus, the term “alkyl” includes for example, trifluoromethyl and trifluoroethyl, among other halogenated alkyls, and hydroxymethyl and other hydroxylated alkyls.

As used herein, the term “alkenyl” (and “alkylene”) refers to straight or branched hydrocarbon chains containing from 2 to 8 carbon atoms, unless a different number of atoms is specified, and at least one and up to three carbon-carbon double bonds. Examples of “alkenyl” as used herein include, but are not limited to ethenyl and propenyl. Examples of “alkenylene” as used herein include, but are not limited to, ethenylene, propenylene and butenylene. “Alkenyl” (and “alkenylene”) also includes substituted alkenyl. The alkenyl groups may optionally be substituted one or more times with a halogen or hydroxyl.

As used herein, the term “alkynyl” refers to straight or branched hydrocarbon chains containing from 2 to 8 carbon atoms, unless a different number of atoms is specified, and at least one and up to three carbon-carbon triple bonds. Examples of “alkynyl” as used herein include, but are not limited to ethynyl and propynyl. “Alkynyl” also includes substituted alkynyl. The alkynyl groups may optionally be substituted one or more times with a halogen or hydroxyl.

As used herein, the term “cycloalkyl” refers to a saturated monocyclic carbocyclic ring having from 3 to 8 carbon atoms, unless a different number of atoms is specified. “Cycloalkyl” includes by way of example cyclopropyl, cyclobutyl, cyclopentyl, cyclohexyl, cycloheptyl and cyclooctyl. “Cycloalkyl” also includes substituted cycloalkyl. The cycloalkyl may optionally be substituted on any available carbon with one or more substituents selected from the group consisting of alkoxy, halo, and haloalkyl, e.g., perfluoroalkyl.

The term “halo” or “halogen” refers to fluoro, chloro, bromo and iodo.

As used herein, the term “alkoxy” refers to the group —O-alkyl, where alkyl is as defined above. Examples of “alkoxy” as used herein include, but are not limited to, methoxy, ethoxy, n-propoxy, isopropoxy, n-butoxy, and t-butoxy. “Alkoxy” also includes substituted alkoxy. The alkoxy groups may be optionally substituted one or more times with a halogen.

As used herein, the term “phenalkoxy” refers to the group —O-alkyl-phenyl, where alkyl is as defined above. Examples of “phenalkoxy” as used herein include, but are not limited to, phenmethoxy and phenethoxy.

The term “aryl” refers to monocyclic carbocyclic groups and fused bicyclic carbocyclic groups having from 6 to 10 carbon atoms, unless a different number of atoms is specified, and having at least one aromatic ring. Examples of particular aryl groups include but are not limited to phenyl and naphthyl. One particular aryl group according to the invention is phenyl.

The present invention provides a compound of formula (I):

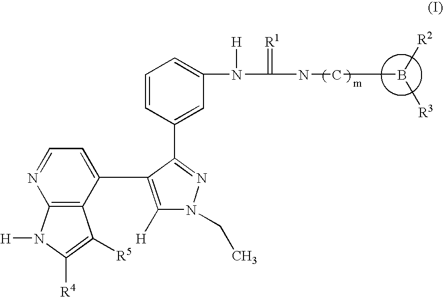

or a pharmaceutically acceptable salt or solvate thereof wherein:
R¹ is selected from O and S;
m is 0 or 1; B is a 6 membered cycloalkyl or aryl ring; R² and R³ are independently selected from H, alkoxy, haloalkyl, halo, and phenalkoxy; R⁴ is selected from H, —C(O)OH, —C(O)—O—CH₂—CH₃; and R⁵ is selected from H and halo.

According to an embodiment of the invention, R¹ is O. According to another embodiment, R¹ is S.

According to another embodiment, both R² and R³ are H.

According to another embodiment, at least one of R² and R³ is selected from alkoxy, haloalkyl, halo, and phenalkoxy.

According to another embodiment, at least one of R² and R³ is halo.

According to another embodiment, at least one of R² and R³ is trifluoroalkyl.

According to another embodiment, at least one of R² and R³ is trifluoromethyl.

According to another embodiment, both R⁴ and R⁵ are H.

According to another embodiment, R⁴ is —C(O)OH.

According to another embodiment, R⁴ is —C(O)—O—CH₂—CH₃.

According to another embodiment, R⁵ is halo.

It is recognized that the compounds of formula (II) are a subset of the compounds of formula I. Therefore, all general statements made and embodiments described herein with regard to the compounds of formula (I) are applicable to the compounds of formula (II).

It is to be understood that the present invention includes all combinations and subsets of the particular groups defined hereinabove.

According to another embodiment of the invention, the invention includes:
N-{3-[1-Ethyl-4-(1H-pyrrolo[2,3-b]pyridin-4-yl)-1H-pyrazol-3-yl]phenyl}-N-phenylurea;
N-(3-Chlorophenyl)-N-{3-[1-ethyl-4-(1H-pyrrolo[2,3-b]pyridin-4-yl)-1H-pyrazol-3-yl]phenyl}urea;
N-{3-[1-Ethyl-4-(1H-pyrrolo[2,3-b]pyridin-4-yl)-1H-pyrazol-3-yl]phenyl}-N-[2-fluoro-5-(trifluoromethyl)phenyl]urea;
N-[4-Chloro-3-(trifluoromethyl)phenyl]-N-{3-[1-ethyl-4-(1H-pyrrolo[2,3-b]pyridin-4-yl)-1H-pyrazol-3-yl]phenyl}urea;
N-{3-[1-Ethyl-4-(1H-pyrrolo[2,3-b]pyridin-4-yl)-1H-pyrazol-3-yl]phenyl}-N-[3-(methyloxy)phenyl]urea;
N-(2,6-Difluorophenyl)-N-{3-[1-ethyl-4-(1H-pyrrolo[2,3-b]pyridin-4-yl)-1H-pyrazol-3-yl]phenyl}urea;
N-{3-[1-Ethyl-4-(1H-pyrrolo[2,3-b]pyridin-4-yl)-1H-pyrazol-3-yl]phenyl}-N-{4-[(phenylmethyl)oxy]phenyl}urea;
N-Cyclohexyl-N-{3-[1-ethyl-4-(1H-pyrrolo[2,3-b]pyridin-4-yl)-1H-pyrazol-3-yl]phenyl}urea;
N-{3-[1-Ethyl-4-(1H-pyrrolo[2,3-b]pyridin-4-yl)-1H-pyrazol-3-yl]phenyl}-N-(phenylmethyl)urea;
N-{3-[1-Ethyl-4-(1H-pyrrolo[2,3-b]pyridin-4-yl)-1H-pyrazol-3-yl]phenyl}-N-[4-(trifluoromethyl)phenyl]ᵤ rea;
N-(2-Chlorophenyl)-N-{3-[1-ethyl-4-(1H-pyrrolo[2,3-b]pyridin-4-yl)-1H-pyrazol-3-yl]phenyl}urea;
N-{3-[1-Ethyl-4-(1H-pyrrolo[2,3-b]pyridin-4-yl)-1H-pyrazol-3-yl]phenyl}-N-phenylthiourea;
N-{3-[4-(3-Chloro-1H-pyrrolo[2,3-b]pyridin-4-yl)-1-ethyl-1H-pyrazol-3-yl]phenyl}-N-phenylurea;
N-{3-[4-(3-Bromo-1H-pyrrolo[2,3-b]pyridin-4-yl)-1-ethyl-1H-pyrazol-3-yl]phenyl}-N-phenylurea;
Ethyl 4-(1-ethyl-3-{3-[({[4-(trifluoromethyl)phenyl]amino}carbonyl)amino]phenyl}-1H-pyrazol-4-yl)-1H-pyrrolo[2,3-b]pyridine-2-carboxylate;
4-(1-Ethyl-3-{3-[({[4-(trifluoromethyl)phenyl]amino}carbonyl)amino]phenyl}-1H-pyrazol-4-yl)-1H-pyrrolo[2,3-b]pyridine-2-carboxylic acid; and
Ethyl 4-(1-ethyl-3-{3-[({[4-(trifluoromethyl)phenyl]amino}carbonyl)amino]phenyl}-1H-pyrazol-4-yl)-1H-pyrrolo[2,3-b]pyridi ne-2-carboxylate.

Specific examples of compounds of the present invention include those recited in the Examples which follow and pharmaceutically acceptable salts or solvates thereof.

It will be appreciated by those skilled in the art that the compounds of the present invention may be utilized in the form of a pharmaceutically acceptable salt or solvate. The pharmaceutically acceptable salts of the compounds of formula (I) include conventional salts formed from pharmaceutically acceptable (i.e., non-toxic) inorganic or organic acids or bases as well as quaternary ammonium salts. Representative salts include the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. Other salts, such as oxalic, which are not themselves pharmaceutically acceptable, may be useful in the preparation of salts useful as intermediates in obtaining compounds of this invention and these form a further aspect of the invention.

The term “solvate” as used herein refers to a complex of variable stoichiometry formed by a solute (a compound of formula (I)) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

Processes for preparing pharmaceutically acceptable salts and solvates of compounds such as the compounds of formula (I) are conventional in the art. See, e.g., Burger's Medicinal Chemistry And Drug Discovery 5th Edition, Vol 1: Principles And Practice.

As will be apparent to those skilled in the art, in the processes described below for the preparation of compounds of formula (I) certain intermediates may be in the form of pharmaceutically acceptable salts or solvates of the compound. Those terms as applied to any intermediate employed in the process of preparing compounds of formula (I) have the same meanings as noted above with respect to compounds of formula (I). Processes for preparing pharmaceutically acceptable salts and solvates of intermediates are known in the art and are analogous to the process for preparing pharmaceutically acceptable salts and solvates of compounds such as the compounds of formula (I).

The compounds of this invention may be in crystalline or non-crystalline form, and, if crystalline, may optionally be hydrated or solvated. This invention includes within its scope stoichiometric hydrates as well as compounds containing variable amounts of water.

Certain compounds of formula (I) may exist in stereoisomeric forms (e.g. they may contain one or more asymmetric carbon atoms or may exhibit cis-trans isomerism). The individual stereoisomers (enantiomers and diastereomers) and mixtures of these are included within the scope of the present invention. The present invention also covers the individual isomers of the compounds represented by formula (I) as mixtures with isomers thereof in which one or more chiral centres are inverted. Certain compounds of formula (I) may be prepared as a mixture of regioisomers. The present invention covers both the mixture of regioisomers as well as the individual compounds. Likewise, it is understood that compounds of formula (I) may exist in tautomeric forms other than that shown in the formula and these are also included within the scope of the present invention.

Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

The compounds of the present invention are inhibitors of one or more B-Raf family kinases. By “B-Raf inhibitor” is meant a compound which exhibits a pIC₅₀ of greater than about 5.5 against at least one B-Raf family kinase in the B-Raf inhibition enzyme assay described below and/or an IC₅₀ of at least about 6.0 μM potency against at least one cell line that overexpresses at least one B-Raf family kinase in the cellular assay described below. In a more particular embodiment “B-Raf inhibitor” refers to a compound which exhibits a pIC₅₀ of greater than about 6.0 against at least one B-Raf family kinase in the B-Raf inhibition enzyme assay described below and/or an IC₅₀ of at least 1.0 μM potency against at least one cell line that overexpresses at least one B-Raf family kinase in the cellular assay described below.

The present invention is not limited to compounds of formula (I) which are selective for B-Raf family kinases; rather, the present invention expressly contemplates compounds of formula (I) which may possess activity against kinases other than B-Raf family kinases, as well. For instance, several compounds of the present invention also possess activity against one or more of Aurora, EGFR, and Erb-B kinasases.

The present invention further provides compounds of formula (I) for use in medical therapy in a mammal, e.g. a human. In particular, the present invention provides compounds of formula (I) for use in the treatment of a condition mediated by at least one B-Raf family kinase in a mammal, and, advantageously, conditions mediated by inappropriate activity of one or more B-Raf family kinase in a mammal. In one embodiment, the present invention provides compounds of formula (I) for use in the treatment of a condition mediated by at least two B-Raf family kinases, and more particularly conditions mediated by inappropriate activity of one or more B-Raf family kinase in a mammal.

The inappropriate B-Raf family kinase activity referred to herein is any B-Raf kinase activity that deviates from the normal B-Raf family kinase activity expected in a particular mammalian subject. Inappropriate B-Raf family kinase activity may take the form of, for instance, an abnormal increase in activity, or an aberration in the timing and/or control of B-Raf family kinase activity. Such inappropriate activity may result then, for example, from overexpression or mutation of the protein kinase or ligand leading to inappropriate or uncontrolled activation of the receptor. Furthermore, it is also understood that unwanted B-Raf family kinase activity may reside in an abnormal source, such as a malignancy. That is, the level of B-Raf family activity does not have to be abnormal to be considered inappropriate, rather the activity derives from an abnormal source.

The compounds of formula (I) and salts and solvates thereof, are believed to have anticancer and antitumor activity as a result of inhibition of one or more B-Raf family protein kinase and its effect on selected cell lines whose growth is dependent on B-Raf family protein kinase activity.

The compounds of formula (I) and salts and solvates thereof, are believed to have activity in treating neurotraumatic diseases as a result of inhibition of one or more B-Raf family protein kinase and its effect on selected cell lines whose growth is dependent on B-Raf family protein kinase activity.

The present invention provides compounds of formula (I) for use in the treatment of a susceptible neoplasm. “Susceptible neoplasm” as used herein refers to neoplasms which are susceptible to treatment with a B-Raf inhibitor. Neoplasms which have been associated with inappropriate activity of one or more B-Raf family kinases and are therefor susceptible to treatment with a B-Raf inhibitor are known in the art, and include both primary and metastatic tumors and cancers. For example, susceptible neoplasms within the scope of the present invention include but are not limited to breast cancer, colon cancer, non-small cell lung cancer, prostate cancer, bladder cancer, ovarian cancer, gastric cancer, pancreatic cancer, carcinoma of the head and neck, esophageal carcinoma, melanoma and renal carcinoma.

The present invention also provides compounds of formula (I) for use in the treatment of a susceptible neurotraumatic disease. Neurotraumatic diseases/events as defined herein include both open or penetrating head trauma, such as caused by surgery, or a closed head trauma injury, such as caused by an injury to the head region. Also included within this definition is ischemic stroke, particularly to the brain area, transient ischemic attacks following coronary by-pass and cognitive decline following other transient ischemic conditions.

The present invention provides methods for the treatment of several conditions in a mammal in need thereof, all of which comprise the step of administering a therapeutically effective amount of a compound of formula (I). The mammal in need of treatment with a compound of the present invention is advantageously a human. According to one embodiment, the condition mediated by at least one B-Raf family kinase is a susceptible neoplasm. According to another embodiment, the condition mediated by at least one B-Raf family kinase is a neurotraumatic disease/event.

As used herein, the term “treatment” refers to alleviating the specified condition, eliminating or reducing the symptoms of the condition, slowing or eliminating the progression of the condition and preventing or delaying the reoccurrence of the condition in a previously afflicted subject.

As used herein, the term “therapeutically effective amount” means an amount of a compound of formula (I) which is sufficient, in the subject to which it is administered, to elicit the biological or medical response of a cell culture, tissue, system, mammal (including human) that is being sought, for instance, by a researcher or clinician. The term also includes within its scope amounts effective to enhance normal physiological function. For example, a therapeutically effective amount of a compound of formula (I) for the treatment of a condition mediated by at least one B-Raf family kinase is an amount sufficient to treat the condition in the subject. Similarly, a therapeutically effective amount of a compound of formula (I) for the treatment of a susceptible neoplasm is an amount sufficient to treat the susceptible neoplasm in the subject. In one embodiment of the present invention, a therapeutically effective amount of a compound of formula (I) is an amount sufficient to regulate, modulate, bind or inhibit at least one B-Raf family kinase.

The precise therapeutically effective amount of the compounds of formula (I) will depend on a number of factors including, but not limited to, the age and weight of the subject being treated, the precise condition requiring treatment and its severity, the nature of the formulation, and the route of administration, and will ultimately be at the discretion of the attendant physician or veterinarian. Typically, the compound of formula (I) will be given for treatment in the range of 0.1 to 200 mg/kg body weight of recipient (mammal) per day and more usually in the range of 1 to 100 mg/kg body weight per day. Acceptable daily dosages, may be from about 0.1 to about 2000 mg/day, and preferably from about 0.1 to about 100 mg/day. Thus, for a 70 kg adult human being treated for a condition mediated by at least one B-Raf family kinase, the actual amount per day would usually be from 70 to 700 mg and this amount may be given in a single dose per day or more usually in a number (such as two, three, four, five or six) of sub-doses per day such that the total daily dose is the same. A therapeutically effective amount of a salt or solvate, may be determined as a proportion of the therapeutically effective amount of the compound of formula (I) per se. It is envisaged that similar dosages would be appropriate for treatment of the other conditions referred to above.

The present invention also provides the use of a compound of formula (I) for the preparation of a medicament for the treatment of condition mediated by at least one B-Raf family kinase in a mammal (e.g., a human) in need thereof. The present invention further provides the use of a compound of formula (I) for the preparation of a medicament for the treatment of a susceptible neoplasm in a mammal.

While it is possible that, for use in therapy, a therapeutically effective amount of a compound of formula (I) may be administered as the raw chemical, it is typically presented as the active ingredient of a pharmaceutical composition or formulation. Accordingly, the invention further provides a pharmaceutical composition comprising a compound of the formula (I). The pharmaceutical composition may further comprise one or more pharmaceutically acceptable carriers, diluents, and/or excipients. The carrier(s), diluent(s) and/or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the formula (I) with one or more pharmaceutically acceptable carriers, diluents and/or excipients.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The compounds of formula (I) can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of formula (I) may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavouring agents.

In the above-described methods of treatment and uses, a compound of formula (I) may be employed alone, in combination with one or more other compounds of formula (I), or in combination with other therapeutic agents. In methods of treating susceptible neoplasms, combination with other chemotherapeutic, hormonal and/or antibody agents is envisaged as well as combination with surgical therapy and radiotherapy. “Chemotherapeutic” agents include but are not limited to anti-neoplastic agents, analgesics and anti-emetics. Anti-emetics include but are not limited to 5HT₃ antagonists such as ondansetron, granisetron, and the like; metaclopromide; dexamethasone and neurokinin-1 antagonists.

As used herein, “anti-neoplastic agents” include both cytostatic and cytotoxic agents. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphor-ines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors.

The compounds of the formula (I) and at least one additional cancer treatment therapy may be employed in combination concomitantly or sequentially in any therapeutically appropriate combination with such other anti-cancer therapies.

In methods of treating susceptible neurotraumas, combination with other neurotrauma treatments is envisaged as well as combination with surgical therapy.

Compounds of formula (I) may be prepared using the processes described below. In all of the schemes described below, it is understood that protecting groups may be employed where necessary in accordance with general principles known to those of skill in the art, for example, see T. W. Green and P. G. M. Wuts (1991) Protecting Groups in Organic Synthesis, John Wiley & Sons. These groups may be removed at a convenient stage of the compound synthesis using methods known to those of skill in the art. The selection of processes as well as the reaction conditions and order of their execution shall be consistent with the preparation of compounds of formula (I).

Compounds of formula (I) may be conveniently prepared by the processes outlined in Schemes 1-5 below. In each of Schemes 1-5, all variables are as defined above. Each of Schemes 1-5 utilize an intermediate (II) prepared according to international patent application WO2002/088107. Compound (IV) can be readily prepared utilizing commercially available compounds by those of ordinary skill according to Organic Lett. 2003, 5, 5023.

For convenience, the group

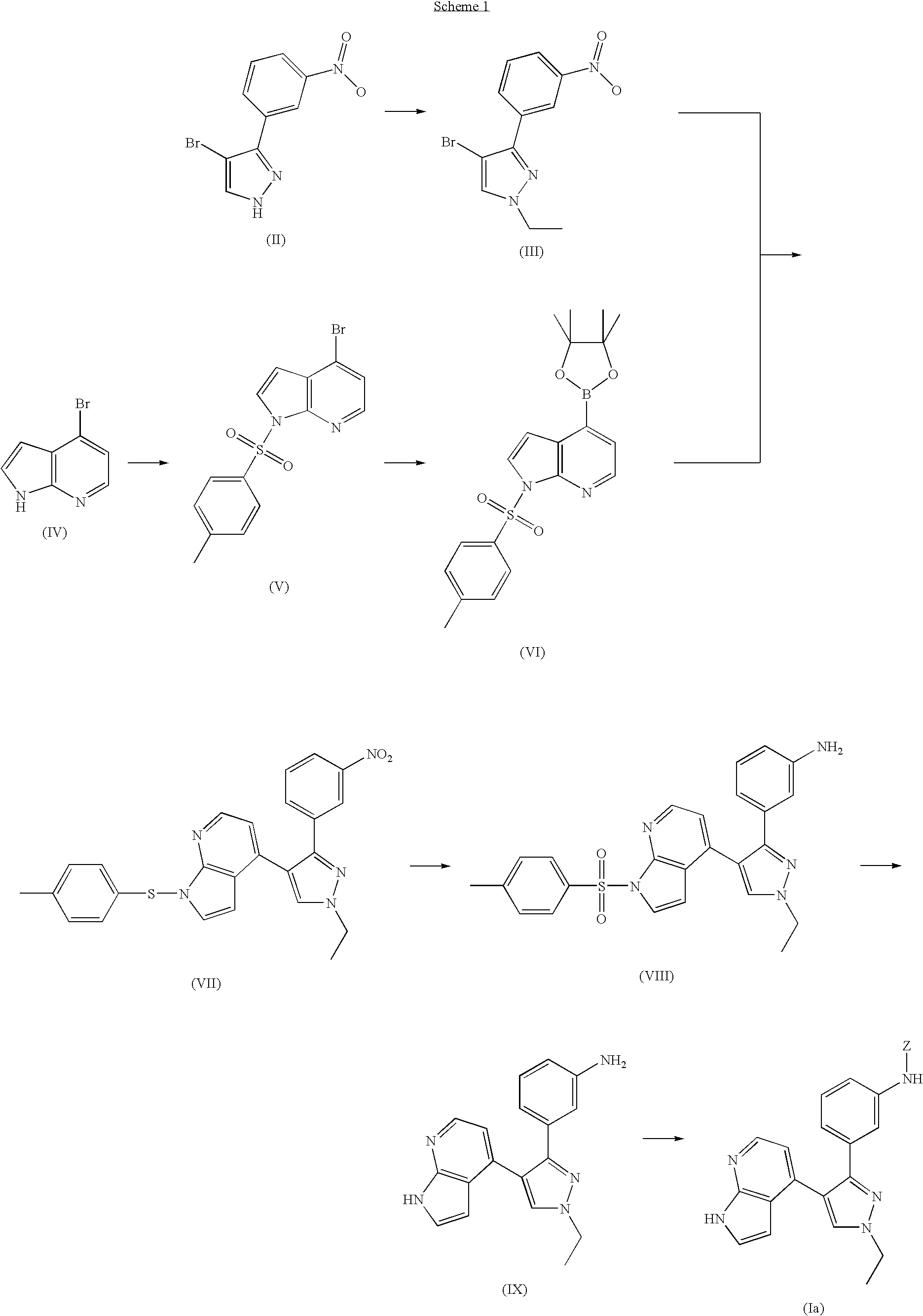

As illustrated in Scheme 1, compounds of formula (Ia) (shown here as the compound of formula I where R⁴ and R⁵ are H, and Z is

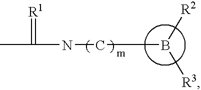

where R¹, m, B, R², and R³ are as defined in the specification above) may be synthesized from compound (IX) by adding R—NCO or R—NCS to a solution of compound (IX) in pyridine, with the molar ratio of compound (IX) to compound R—NCO or R—NCS being from 1:1 to 1:1.5, preferably 1:1.2. While the preferred solvent is pyridine, those skilled in the art will understand that other solvents, such as CH₂Cl₂, CHCl₃, CH₂ClCH₂Cl, can be used. The reaction mixture is stirred at room temperature for 1 hour. As will be understood by those skilled in the art, various temperatures such as 0 to 60° C. and times such as 0.1 to 12 hours can be used. Compounds of formula (I) can be obtained by concentration in vacuo and purification by mass directed LC/MS, for example.

The tosyl group illustrated as a nitrogen-protecting group in Scheme 1 may be removed, for example, by treatment with aqueous NaOH, or other methods known to one skilled in the art. Other suitable nitrogen protecting groups may also be employed in this synthetic scheme. For example, reaction of compound (VIII), which can be prepared according to description in Scheme 1, with aqueous NaOH in an appropriate solvent such as MeOH at 40° C. for 1 h yields compound (IX).

Compounds of formula (VIII) can be prepared by the reduction of compound (VII) with a reductant in an appropriate solvent at temperatures between rt and 250° C. For example, heating compound (VII) with tin in EtOH and aqueous HCl at reflux for 4 h provides compound (VIII).

To prepare compound (VII), compound (III) and compound (VI), in a molar ratio that is typically 1:1, but can vary from 1:1 to 1:3 are dissolved in DME and 2M aqueous Na₂CO₃, which is typically used in a 36:1 volume ratio, in the presence of a palladium catalyst and heated at elevated temperature. 2M Na₂CO₃ can be used from 0.25 to 10 equivalence with respect to compound (VI). In place of Na₂CO₃, other bases such as K₂CO₃, K₃PO₄, Cs₂CO₃, CsF, Ba(OH)₂, NaOH, NaHCO₃ can be used. Instead of or in addition to DME, other solvent systems such as DMF, dioxane, THF, toluene, xylene, MeOH, ethanol, H₂O, MeCN, NMP or a mixture of two or more of them could be used. The catalyst is preferably Pd(PPh₃)₄; however, it is understood that other catalysts such as Pd(OAc)₂, Pd₂(dba)₃, [PdCl(allyl)]₂, with a suitable ligand such as PPh₃, PCy₃, (t-Bu)₂POH, (t-Bu)₃P could be used. Also, phosphine-free palladium such as Pd/C, and polymer bound palladium may be used. The use of a different catalyst may alter the time, temperature, and/or solvent to be used as will be understood by one skilled in the art. The reaction is preferably performed using microwave heating at 120° C. for 60 minutes. However, other modes of heating such as oil baths or hot plates may also be used. Also, other temperatures of 60 to 180° C. and times of 0.1 to 24 h may be utilized, with the general understanding that higher reaction temperatures typically will require shorter reaction times. The filtrate can be purified by SCX cartridge via capture-and-release, for example.

To prepare compound (VI), compound (V) and commercial available 4,4,4′,4′,5,5,5′,5′-octamethyl-2,2′-bi-1,3,2-dioxaborolane (available from Aldrich), in a molar ratio that is typically 1:2, but can vary from 1:1 to 1:3, and KOAc are dissolved in DMF and heated at elevated temperature in the presence of a palladium catalyst. KOAc can be used from 1 to 10 equivalence with respect to 4,4,4′,4′,5,5,5′,5′-octamethyl-2,2′-bi-1,3,2-dioxaborolane. The catalyst is preferably Pd(dppf)Cl₂. The reaction is preferably performed at 90° C. for 48 h. Also, other temperatures of 60 to 180° C. and times of 0.1 to 100 h may be utilized, with the general understanding that higher reaction temperatures typically will require shorter reaction times.

Compound (V) can be synthesized by standard synthetic methods known to those skilled in the art to be useful for the incorporation of a nitrogen-protecting group. For example, reaction of compound (IV) with tosyl chloride and catalytic amount of Bu₄NHSO₄ in a co-solvent such as CH₂Cl₂ and aqueous NaOH at rt for 30 minutes yields compound (V).

Compound (III) can be synthesized by reaction of compound (II) with ethyl iodide in a solvent such as dichloromethane in the presence of an appropriate base such as aqueous NaOH. Addition of tetrabutylammonium bromide is also preferred. For example, stirring compound (II) with Etl, TBAB at rt for 1 h provides compound (III).

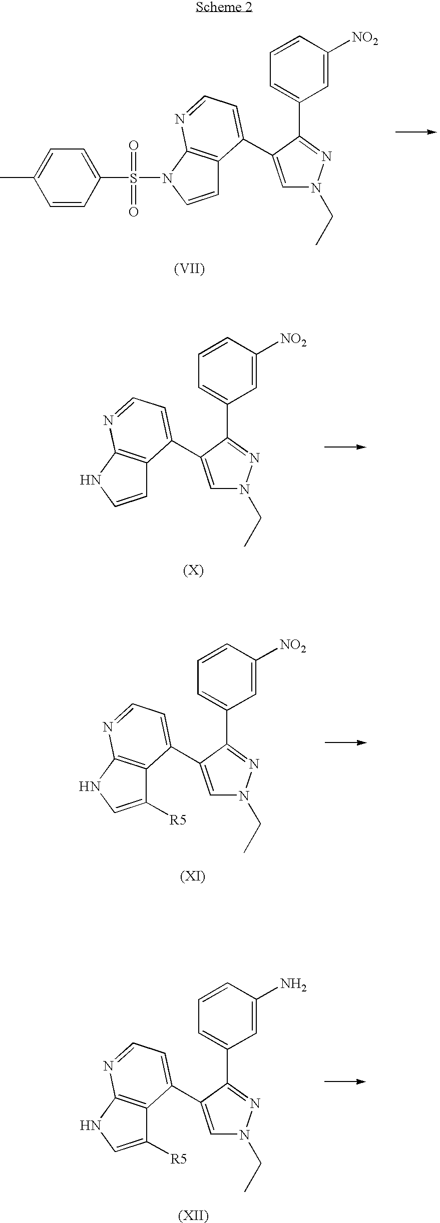

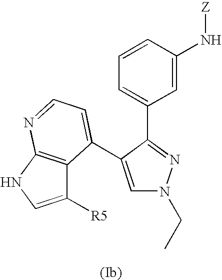

As illustrated in Scheme 2, compounds of formula (Ib) (shown here as the compound of formula I where R⁵ is a halogen and Z is as described in Scheme 1) may be synthesized from compound (XII) by adding R—NCO or R—NCS to a solution of compound (XII) in pyridine, with the molar ratio of compound (XII) to compound R—NCO or R—NCS being from 1:1 to 1:1.5, preferably 1:1.2. While the preferred solvent is pyridine, those skilled in the art will understand that other solvents, such as CH₂Cl₂, CHCl₃, CH₂ClCH₂Cl, can be used. The reaction mixture is stirred at room temperature for 1 hour. As will be understood by those skilled in the art, various temperatures such as 0 to 60° C. and times such as 0.1 to 12 hours can be used. Compound (I) can be obtained by concentration in vacuo and purification by mass directed LC/MS.

Compounds of formula (XII) can be prepared by the reduction of compound (XI) with an appropriate reductant in an appropriate solvent at temperatures between rt and 250° C. For example, heating compound (XI) with tin in EtOH and aqueous HCl at reflux for 12 hours provides compound (XII).

To prepare compound (XI), NBS or NCS is added to a solution of compound (X), in an appropriate solvent such as THF, with the molar ratio of compound (X) to NBS or NCS being from 1:1 to 1:1.5, preferably 1:1.15. While the preferred solvent is THF, those skilled in the art will understand that other solvents, such as CH₂Cl₂, CHCl₃, CH₂ClCH₂Cl, CCl₄, dioxane can be used. The reaction mixture is stirred at room temperature for 12 hours. As will be understood by those skilled in the art, various temperatures such as 0 to 60° C. and times such as 0.1 to 12 hours can be used. Compound (XI) can be obtained by concentration in vacuo and purification by mass directed LC/MS, for example.

The tosyl group illustrated as a nitrogen-protecting group in Scheme 2 may be removed, for example, by treatment with aqueous NaOH, or other methods known to one skilled in the art. Other suitable nitrogen protecting groups may also be employed in this synthetic scheme. For example, reaction of compound (VII), which can be prepared according to description in Scheme 1, with aqueous NaOH in a solvent such as MeOH at rt for 12 h yields compound (X).

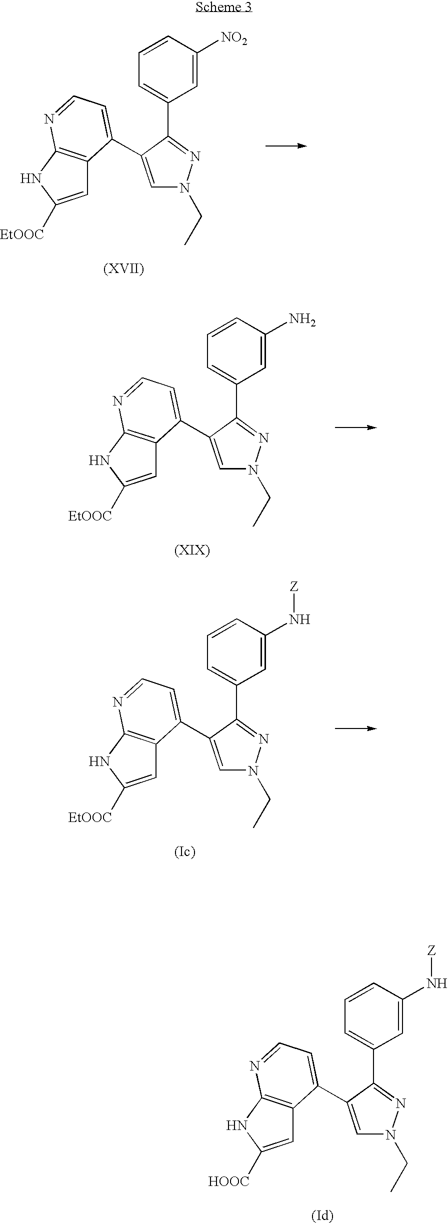

Compound (Id) (shown here as the compound of formula I where R⁴ is HOOC and Z is as defined in Scheme I), as illustrated in Scheme 3, can be synthesized by standard synthetic methods known to those skilled in the art to be useful for the removal of the ethyl group from ester in compound (Ic). For example, heating the reaction of compound (Ic) (shown here as the compound of formula I where R⁴ is EtOOC and Z is as defined in Scheme 1) with aqueous NaOH in an appropriate solvent such as MeOH at reflux for 1 h yields compound (Id).

As illustrated in Scheme 3, compounds of formula (XX) may be synthesized from compound (XIX) by adding R—NCO or R—NCS to a solution of compound (XIX) in pyridine, with the molar ratio of compound (XIX) to compound R—NCO or R—NCS being from 1:1 to 1:1.5, preferably 1:1.2. While the preferred solvent is pyridine, those skilled in the art will understand that other solvents, such as CH₂Cl₂, CHCl₃, CH₂ClCH₂Cl, can be used. The reaction mixture is stirred at room temperature for 1 hour. As will be understood by those skilled in the art, various temperatures such as 0 to 60° C. and times such as 0.1 to 12 hours can be used.

Compound (XIX) can be prepared by the reduction of compound (XVII) with an appropriate reductant in an appropriate solvent at temperatures between rt and 250° C. For example, heating compound (XVII) with tin in EtOH and aqueous HCl at reflux for 4 hours provides compound (XIX).

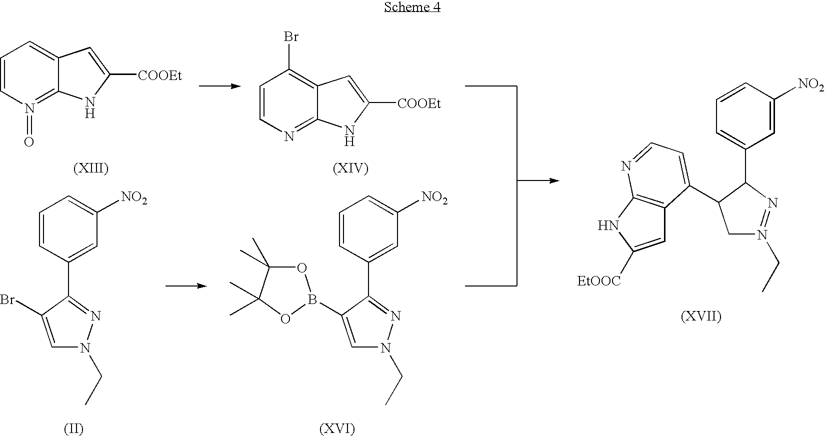

To prepare compound (XVII), compound (XIV) and compound (XVI), in a molar ratio that is typically 1:1, but can vary from 1:1 to 1:3 are dissolved in DME and 2M aqueous Na₂CO₃, which is typically used in a 36:1 volume ratio, in the presence of a palladium catalyst and heated at elevated temperature. 2M Na₂CO₃ can be used from 0.25 to 10 equivalence with respect to compound (XVI). In place of Na₂CO₃, other bases such as K₂CO₃, K₃PO₄, Cs₂CO₃, CsF, Ba(OH)₂, NaOH, NaHCO₃ could be used. Instead of or in addition to DME, other solvent systems such as DMF, dioxane, THF, toluene, xylene, MeOH, ethanol, H₂O, MeCN, NMP or a mixture of two or more of them could be used. The catalyst is preferably Pd(PPh₃)₄; however, it is understood that other catalysts such as Pd(OAc)₂, Pd₂(dba)₃, [PdCl(allyl)]₂, with a suitable ligand such as PPh₃, PCy₃, (t-Bu)₂POH, (t-Bu)₃P could be used. Also, phosphine-free palladium such as Pd/C, and polymer bound palladium may be used. The use of a different catalyst may alter the time, temperature, and/or solvent to be used as will be understood by one skilled in the art. The reaction is preferably performed using microwave heating at 120° C. for 30 minutes. However, other modes of heating such as oil baths or hot plates may also be used. Also, other temperatures of 60 to 180° C. and times of 0.1 to 24 h may be utilized, with the general understanding that higher reaction temperatures typically will require shorter reaction times. The filtrate can be purified by SCX cartridge via capture-and-release, for example.

To prepare compound (XIV), methanesulfonic anhydride is added to a solution of compound (XIII) and tetramethylammonium bromide, in an appropriate solvent such as DMF, with the molar ratio of compound (XIII) to tetramethylammonium bromide and methanesulfonic anhydride being from 1:1:1 to 1:2:4, preferably 1:1.5:2. While the preferred solvent is DMF, those skilled in the art will understand that other solvents, such as DMSO can be used. The reaction mixture is standing from 0° C. to room temperature for 6 hours. As will be understood by those skilled in the art, various temperatures such as 0 to 60° C. and times such as 0.1 to 12 hours can be used.

Compound (XIII) can be readily prepared according to international patent application WO2000/044753.

To prepare compound (XVI), compound (II) and commercial available 4,4,4′,4′,5,5,5′,5′-octamethyl-2,2′-bi-1,3,2-dioxaborolane, in a molar ratio that is typically 1:2, but can vary from 1:1 to 1:3, and KOAc are dissolved in DMF and heated at elevated temperature in the presence of a palladium catalyst. KOAc can be used from 1 to 10 equivalence with respect to 4,4,4′,4′,5,5,5′,5′-octamethyl-2,2′-bi-1,3,2-dioxaborolane. The catalyst is preferably Pd(dppf)Cl₂. The reaction is preferably performed at 90° C. for 12 h. Also, other temperatures of 60 to 180° C. and times of 0.1 to 100 hours may be utilized, with the general understanding that higher reaction temperatures typically will require shorter reaction times.

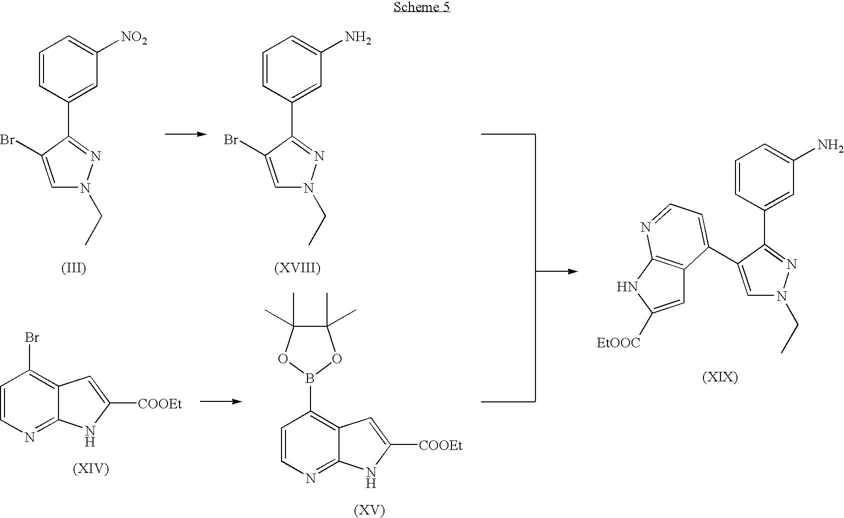

To prepare compound (XIX), another procedure can also be used as illustrated in Scheme 5. Compound (XVIII) and compound (XV), in a molar ratio that is typically 1:1, but can vary from 1:1 to 1:3 are dissolved in DME and 2M aqueous Na₂CO₃, which is typically used in a 36:1 volume ratio, in the presence of a palladium catalyst and heated at elevated temperature. 2M Na₂CO₃ could be used from 0.25 to 10 equivalence with respect to compound (XV). In place of Na₂CO₃, other bases such as K₂CO₃, K₃PO₄, Cs₂CO₃, CsF, Ba(OH)₂, NaOH, NaHCO₃ could be used. Instead of or in addition to DME, other solvent systems such as DMF, dioxane, THF, toluene, xylene, MeOH, ethanol, H₂O, MeCN, NMP or a mixture of two or more of them could be used. The catalyst is preferably Pd(PPh₃)₄; however, it is understood that other catalysts such as Pd(OAc)₂, Pd₂(dba)₃, [PdCl(allyl)]₂, with a suitable ligand such as PPh₃, PCy₃, (t-Bu)₂POH, (t-Bu)₃P could be used. Also, phosphine-free palladium such as Pd/C, and polymer bound palladium may be used. The use of a different catalyst may alter the time, temperature, and/or solvent to be used as will be understood by one skilled in the art. The reaction is preferably performed using microwave heating at 120° C. for 60 minutes. However, other modes of heating such as oil baths or hot plates may also be used. Also, other temperatures of 60 to 180° C. and times of 0.1 to 24 h may be utilized, with the general understanding that higher reaction temperatures typically will require shorter reaction times. The filtrate can be purified by SCX cartridge via capture-and-release, for example.

To prepare compound (XV), compound (XIV) and commercial available 4,4,4′,4′,5,5,5′,5′-octamethyl-2,2′-bi-1,3,2-dioxaborolane, in a molar ratio that is typically 1:2, but can vary from 1:1 to 1:3, and KOAc are dissolved in DMF and heated at elevated temperature in the presence of a palladium catalyst. KOAc could be used from 1 to 10 equivalence with respect to 4,4,4′,4′,5,5,5′,5′-octamethyl-2,2′-bi-1,3,2-dioxaborolane. The catalyst is preferably Pd(dppf)Cl₂. The reaction is preferably performed at 90° C. for 48 h. Also, other temperatures of 60 to 180° C. and times of 0.1 to 100 hours may be utilized, with the general understanding that higher reaction temperatures typically will require shorter reaction times.

Compound (XIV) can be obtained as illustrated in Scheme 4.

Compound (XVIII) can be prepared by the reduction of compound (III) with an appropriate reductant in an appropriate solvent at temperatures between rt and 250° C. For example, heating compound (III) with tin in EtOH and aqueous HCl at reflux for 4 hours provides compound (XVIII).

The present invention also provides radiolabeled compounds of formula (I) and biotinylated compounds of formula (I) and solid-support-bound versions thereof. Radiolabeled compounds of formula (I) and biotinylated compounds of formula (I) can be prepared using conventional techniques. For example, radiolabeled compounds of formula (I) can be prepared by reacting the compound of formula (I) with tritium gas in the presence of an appropriate catalyst to produce radiolabeled compounds of formula (I).

In one embodiment, the compounds of formula (I) are tritiated.

The radiolabeled compounds of formula (I) and biotinylated compounds of formula (I) are useful in assays for the identification of compounds which inhibit at least one B-Raf family kinase, for the identification of compounds for the treatment of a condition mediated by at least one B-Raf family kinase, for the treatment of susceptible neoplasms. Accordingly, the present invention provides an assay method for identifying such compounds, which method comprises the step of specifically binding the radiolabeled compound of formula (I) or the biotinylated compound of formula (I) to the target protein or cellular homogenates. More specifically, suitable assay methods will include competition binding assays. The radiolabeled compounds of formula (I) and biotinylated compounds of formula (I) and solid-support-bound versions thereof, can be employed in assays according to the methods conventional in the art.

The following examples are intended for illustration only and are not intended to limit the scope of the invention in any way, the invention being defined by the claims which follow.

### EXAMPLES

As used herein, the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

g (grams); mg (milligrams);

L (liters); mL (milliliters);

μL (microliters); psi (pounds per square inch);

M (molar); mM (millimolar);

i. v. (intravenous); Hz (Hertz);

MHz (megahertz); mol (moles);

mmol (millimoles); rt (room temperature);

min (minutes); h (h);

mp (melting point); TLC (thin layer chromatography);

Tᵣ (retention time); RP (reverse phase);

MeOH (methanol); i-PrOH (isopropanol);

TEA (triethylamine); TFA (trifluoroacetic acid);

TFAA (trifluoroacetic anhydride); THF (tetrahydrofuran);

DMSO (dimethylsulfoxide); AcOEt (EtOAc);

DME (1,2-dimethoxyethane); DCM (CH₂Cl₂);

DCE (dichloroethane); DMF (N,N-dimethylformamide);

DMPU (N,N′-dimethylpropyleneurea); (CDI (1,1-carbonyldiimidazole); IBCF (isobutyl CHCl3ate); HOAc (acetic acid);

HOSu (N-hydroxysuccinimide); HOBT (1-hydroxybenzotriazole);

mCPBA (meta-chloroperbenzoic acid);

BOC (tert-butyloxycarbonyl); FMOC (9-fluorenyl methoxycarbonyl);

DCC (dicyclohexylcarbodiimide); CBZ (benzyloxycarbonyl);

NBS (N-bromosuccinimide); NCS(N-chlorosuccinimide);

Ac (acetyl); atm (atmosphere);

TMSE (2-(trimethylsilyl)ethyl); TMS (trimethylsilyl);

TIPS (triisopropylsilyl); TBS (t-butyldimethylsilyl);

DMAP (4-dimethylaminopyridine); BSA (bovine serum albumin)

ATP (adenosine triphosphate); HRP (horseradish peroxidase);

DMEM (Dulbecco's modified Eagle medium);

HPLC (high pressure liquid chromatography);

BOP (bis(2-oxo-3-oxazolidinyl)phosphinic chloride);

TBAF (tetra-n-butylammonium fluoride);

HBTU (O-Benzotriazole-1-yl-N,N,N′,N′-tetramethyluronium hexafluorophosphate);

TBTU (O-Benzotriazole-1-yl-N,N,N′,N′-tetramethyluronium tetrafluoroborate);

HEPES (4-(2-hydroxyethyl)-1-piperazine ethane sulfonic acid);

DPPA (diphenylphosphoryl azide);

fHNO₃ (fumed HNQ₃);

EDC (ethylcarbodiimide hydrochloride);

EDTA (ethylenediaminetetraacetic acid);

DIEA (N,N-diisopropyl-N-ethylamine);

dppf (1,1′-bis(diphenylphosphino)ferrocene);

R—NCO (an isocyanate);

R—NCS (an isothiocyanate); and

NMP (1-methyl-2-pyrrolidinone)

All references to ether are to diethyl ether; brine refers to a saturated aqueous solution of NaCl. Unless otherwise indicated, all temperatures are expressed in ° C. (degrees Centigrade). All reactions are conducted under an inert atmosphere at room temperature unless otherwise noted.

¹H NMR spectra were recorded on a Varian VXR-300, a Varian Unity-300, a Varian Unity-400 instrument, a Brucker AVANCE-400, or a General Electric QE-300. Chemical shifts are expressed in parts per million (ppm, 6 units). Coupling constants are in units of hertz (Hz). Splitting patterns describe apparent multiplicities and are designated as s (singlet), d (doublet), t (triplet), q (quartet), quint (quintet), m (multiplet), br (broad).

Low-resolution mass spectra (MS) were recorded on a Waters micromass ZQ2000; 2695 Alliance; 2996 Photodiode Array Detector. Preparative LC/MS purification uses the following condition:

### Waters FractionLynx LC/MS Condition

Autosampler/Fractioncollector: 2767 inject collector
Waste collector: waters fraction collector 2
HPLC: 2525 pump
Detector: 2996 Photodiode Array Detector
MS: ZQ2000
Make up pump: waters reagent manager

### Purification Protocol

Loading: 5-100 mg,
4 gradient methods, Cycle time: 15 min
Flow rate: 40 mL/min
Column: XTerraTMMSC₁₈, 30×150 mm (10 μmm)
Injection Volume: 1800 μL
Temperature: rt

### Basic Condition Mobile Phase

A—(Pure H₂O:3 L+28% Ammonia solution 11 mL)

B—100% Acetonitrile

### Acidic Condition Mobile Phase

A—(Pure H₂O:3 L+100% Formic acid 3 mL)

B—(Acetonitrile: 3 L+100% Formic acid 3 mL)

### Make Up Solvent

20% H₂O+80% Methanol+10 mM ammonium acetate
Gradient: 6 gradient methods for purification (Solvent B ratio)

| | | Method | Method | Method | Method | Method | Method |
|---|---|---|---|---|---|---|---|
| Time | flow | 1 | 2 | 3 | 4 | 5 | 6 |
| 0 | 1 | 5 | 5 | 5 | 5 | 5 | 5 |
| 0.5 | 40 | 5 | 5 | 5 | 5 | 5 | 5 |
| 1 | 40 | 5 | 10 | 20 | 35 | 50 | 65 |
| 10 | 40 | 25 | 25 | 40 | 55 | 75 | 90 |
| 13.5 | 40 | 100 | 100 | 100 | 100 | 100 | 100 |
| 15 | 40 | 100 | 100 | 100 | 100 | 100 | 100 |

All mass spectra were taken under electrospray ionization (ESI), chemical ionization (CI), and electron impact (EI) or by fast atom bombardment (FAB) methods. All reactions were monitored by thin-layer chromatography on 0.25 mm E. Merck silica gel plates (60F-254), visualized with UV light, 5% ethanolic phosphomolybdic acid or p-anisaldehyde solution or mass spectrometry (electrospray or AP). Flash column chromatography was performed on silica gel (230-400 mesh, Merck) or using automated silica gel chromatography (Yamazen Fast Flow Liquid Chromatography, UV detection triggering sample collection).

Microwave irradiation was performed on a Personal Chemistry Smithsynthesizer or Creator.

SCX purification: Varian Mega Bond Elut SCX; General procedure: A SCX cartridge was rinsed with MeOH, and then crude mixture was dissolved into a suitable solvent such as MeOH, DCM etc. and loaded on the cartridge. And then the cartridge was rinsed with methanol and dichloromethane successively. The product was isolated by elution with a 2M ammonia solution in methanol (for some cases, mixed with DCM), followed by concentration in vacuo.

### General Intermediate 1

### 4-Bromo-1-ethyl-3-(3-nitrophenyl)-1H-pyrazole

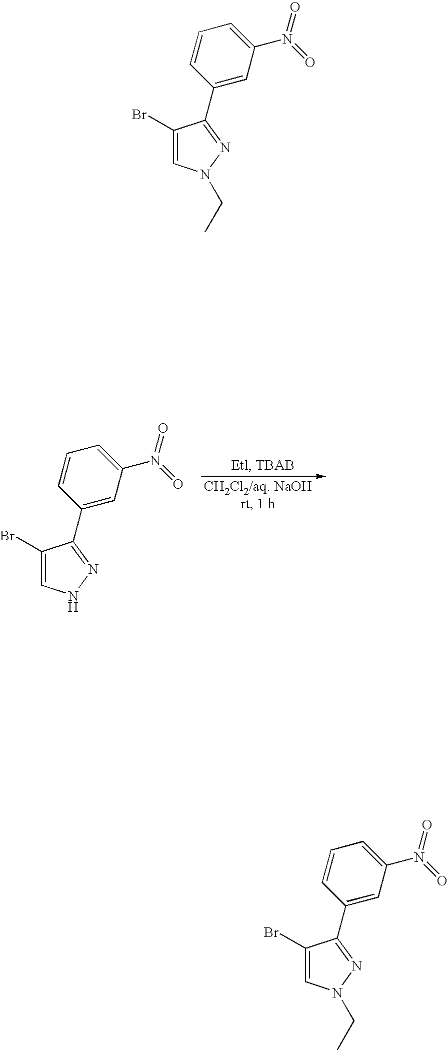

To a solution of 4-bromo-3-(3-nitrophenyl)-1H-pyrazole (7.1 g, 24.1 mmol), which was prepared according to WO2002088107, in dichloromethane (570 mL), was added aqueous NaOH (2N, 114 mL). Then, tetrabutylammonium bromide (9.6 g, 29.8 mmol) was added to the resulting mixture under vigorously stirring, followed by ethyl iodide (6.8 mL). After stirring at rt for 1 h, the organic phase was separated, washed with 0.5 N aqueous HCl (250 mL), 5% aqueous NaHCO₃, and dried over MgSO₄. Concentration in vacuo and purification by column chromatography on silica gel (eluted with hexane/ethyl acetate=7/1) afforded the major regioisomer (5.8 g) as a pale yellow solid. ¹H NMR (400 MHz, CDCl₃) ppm 1.55 (t, 3H, J=7.3 Hz), 4.22 (q, 2H, J=7.3 Hz), 7.54 (s, 1H), 7.59 (t, 1H, J=8.1 Hz), 8.20 (m, 1H), 8.28 (m, 1H), 8.82 (m, 1H). LC/MS: m/z 296, 298 (M+1)⁺.

The other minor regioisomer of 4-bromo-1-ethyl-5-(3-nitrophenyl)-1H-pyrazole (0.58 g) was also isolated as a solid. ¹H NMR (400 MHz, CDCl₃) ppm 1.39 (t, 3H, J=7.3 Hz), 4.13 (q, 2H, J=7.3 Hz), 7.60 (s, 1H), 7.71-7.77 (m, 2H), 8.29 (m, 1H), 8.35 (m, 1H). LC/MS: m/z 296, 298 (M+1)+.

### General Intermediate 2

### 3-(4-Bromo-1-ethyl-1H-pyrazol-3-yl)aniline

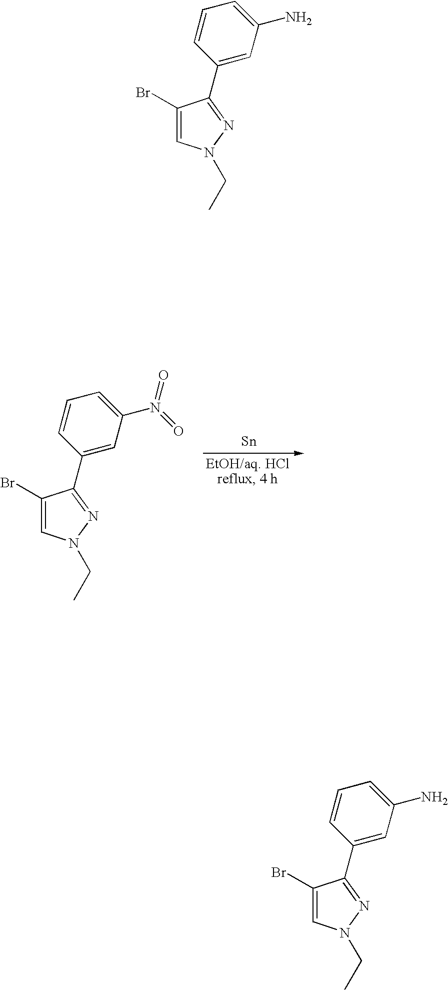

To a suspension of 4-bromo-1-ethyl-3-(3-nitrophenyl)-1H-pyrazole (500 mg, 1.7 mmol) and tin (1.0 g, 8.4 mmol) in EtOH (100 mL), was added 6N aqueous HCl (1 mL). The reaction was refluxed for 4 h, and directly charged into SCX cartridge. After SCX purification, the residue was purified by Yamazen Fast Flow Liquid Chromatography on a silica gel column (EtOAc:Hexane=1:1 to 1:0) to furnish the desired product in 95% yield. ¹H NMR (400 MHz, DMSO-d6) ppm 1.39 (t, 3H, J=7.3 Hz), 4.14 (q, 2H, J=7.3 Hz), 5.16 (brs, 2H), 6.55 (ddd, 1H, J=1.0, 1.8, 7.8 Hz), 6.95 (ddd, 1H, J=1.0, 1.8, 7.8 Hz), 7.02 (dd, 1H, J=1.8, 1.8 Hz), 7.06 (dd, 1H, J=7.8, 7.8 Hz), 8.03 (s, 1H). MS (ESI): m/z 266, 268 (M+1)+

### General Intermediate 3: 1-[(4-Methylphenyl)sulfonyl]-4-(4,4,5,5-tetramethyl-1,3,2-dioxaborolan-2-yl)-1H-pyrrolo[2,3-b]pyridine

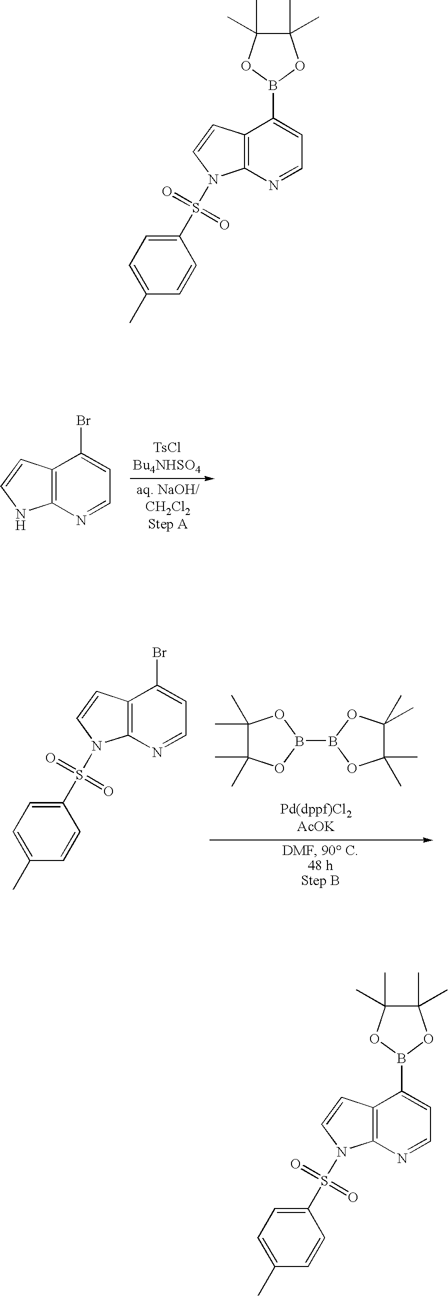

### Step A: 4-Bromo-1-[(4-methyl phenyl)sulfonyl]-1H-pyrrolo[2,3-b]pyridine

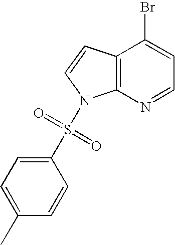

Aqueous NaOH (6N, 5 mL) was added to a solution of 4-bromo-1H-pyrrolo[2,3-b]pyridine (1.6 g, 8.1 mmol), TsCl (3.1 g, 2.0 mmol) and Bu₄NHSO₄ (82.7 mg, 0.3 mmol) in CH₂Cl₂ (40 mL). After stirring the mixture at rt for 30 min, the reaction was quenched by saturated aqueous NH₄Cl, and extracted with CH₂Cl₂ (20 mL×3 times). The organic layer was washed with brine, dried over Na₂SO₄, and then evaporated to dryness under reduced pressure. The residue was purified on a silica gel column (EtOAc:Hex=1:4), and the corresponding product was obtained as a white solid (2.0 g, 70%). ¹H NMR (400 MHz, DMSO-d6) ppm 2.35 (s, 3H), 6.79 (d, 1H, J=4.0 Hz), 7.43 (d, 2H, J=8.1 Hz), 7.61 (d, 1H, J=5.3 Hz), 8.00 (d, 2H, J=8.1 Hz), 8.04 (d, 1H, J=4.0 Hz), 8.25 (d, 1H, J=5.1 Hz). MS (ESI): m/z 351,353 (M+1)+

### Step B: 1-[(4-Methylphenyl)sulfonyl]-4-(4,4,5,5-tetramethyl-1,3,2-dioxaborolan-2-yl)-1H-pyrrolo[2,3-b]pyridine

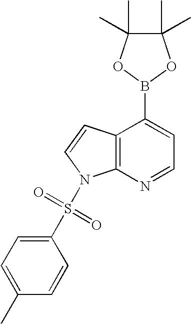

A mixture of 4-bromo-1-[(4-methylphenyl)sulfonyl]-1H-pyrrolo[2,3-b]pyridine (1.9 g, 5.4 mmol), 4,4,4′,4′,5,5,5′,5′-octamethyl-2,2′-bi-1,3,2-dioxaborolane (2.8 g, 10.9 mmol), KOAc (1.6 g, 16.3 mmol) and Pd(dppf)Cl₂ (0.4 g, 0.5 mmol) in DMF (20 mL) was heated at 90° C. for 48 h under inert atmosphere. After cooling to rt, 1 N aqueous NaOH was added till the aqueous layer was taken to pH 10. The aqueous layer was washed with CH₂Cl₂, carefully acidified to pH 4 with 1 N aqueous HCl, and extracted with CH₂Cl₂ (20 mL×3 times). The organic layer was concentrated under reduced pressure to give the desired boronic ester as a brown solid (1.7 g, 77%). ¹H NMR (400 MHz, DMSO-d6) ppm 1.32 (s, 12H), 2.33 (s, 3H), 6.95 (d, 1H, J=4.0 Hz), 7.40 (d, 2H, J=8.1 Hz), 7.47 (d, 1H, J=4.5 Hz), 7.93-7.98 (m, 3H), 8.38 (d, 1H, J=4.5 Hz). MS (ESI): m/z 399 (M+1)+

### General Intermediate 4: 4-[1-Ethyl-3-(3-nitrophenyl)-1H-pyrazol-4-yl]-1-[(4-methylphenyl)sulfonyl]-1H-pyrrolo[2,3-b]pyridine

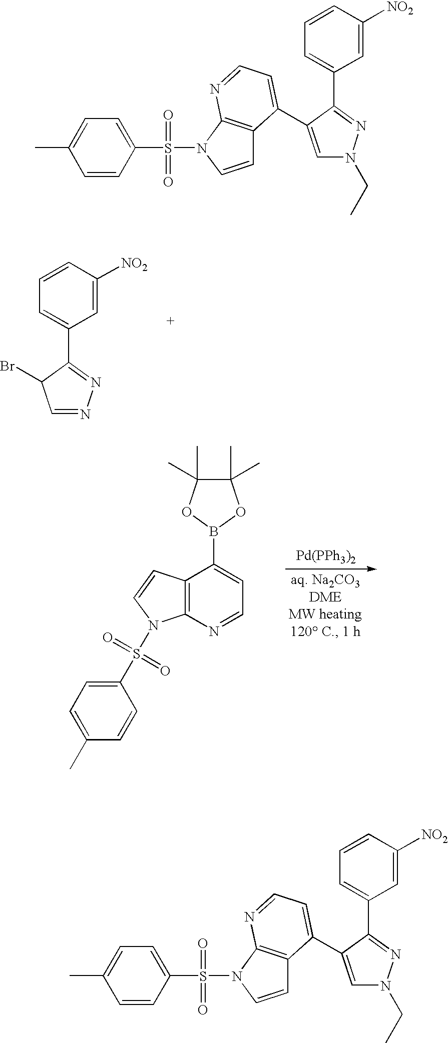

4-Bromo-1-ethyl-3-(3-nitrophenyl)-1H-pyrazole (general intermediate 1, 740.3 mg, 2.5 mmol) and 1-[(4-methylphenyl)sulfonyl]-4-(4,4,5,5-tetramethyl-1,3,2-dioxaborolan-2-yl)-1H pyrrolo[2,3-b]pyridine (general intermediate 3, 1.0 g, 2.5 mmol) were dissolved in DME (18 mL) and aqueous Na₂CO₃ (2M, 0.5 mL) in a microwave vial. Pd(PPh₃)₄ (288.9 mg, 0.25 mmol) was added to the resulting solution. After heating by microwave irradiation with Creator at 120° C. for 1 h, the reaction was diluted with saturated aqueous NH₄Cl, and extracted with CH₂Cl₂ (20 mL×3 times). The organic layer was washed with brine, dried over Na₂SO₄, and then evaporated to dryness under reduced pressure. The residue was purified by silica gel chromatography (EtOAc:Hex=1:4 to 1:1), and the corresponding product was obtained as a white solid (1.1 g, 88%). ¹H NMR (400 MHz, DMSO-d6) ppm 1.49 (t, 3H, J=7.3 Hz), 2.36 (s, 3H), 4.28 (q, 2H, J=7.3 Hz), 6.51 (d, 1H, J=4.0 Hz), 7.08 (d, 1H, J=5.1 Hz), 7.43 (d, 2H, J=8.1 Hz), 7.55 (dd, 1H, J=8.1, 8.1 Hz), 7.65-7.70 (m 1H), 7.84 (d, 1H, J=4.0 Hz), 7.98 (d, 2H, J=8.1 Hz), 8.13-8.18 (m, 2H), 8.27-8.31 (m, 2H). MS (ESI): m/z 488 (M+1)+

### General Intermediate 5

### 3-[1-Ethyl-4-(1H-pyrrolo[2,3-b]pyridin-4-yl)-1H-pyrazol-3-yl]aniline

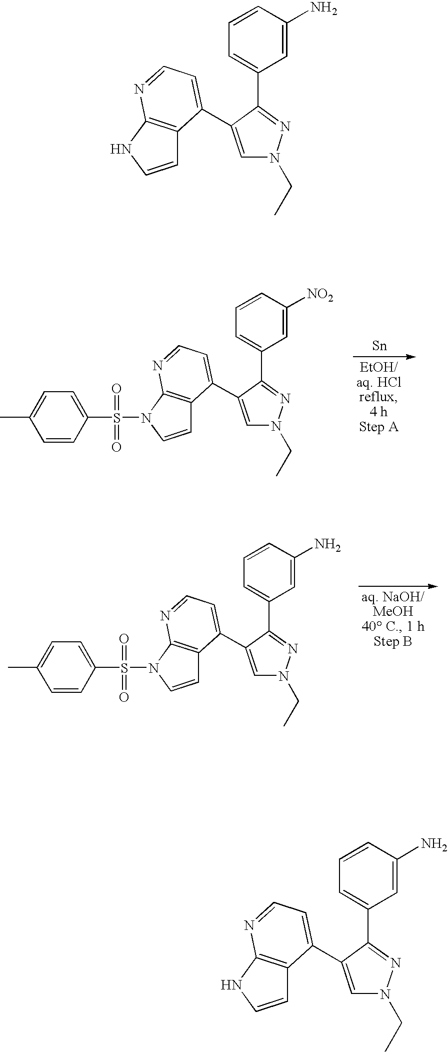

### Step A

### 4-[1-Ethyl-3-(3-nitrophenyl)-1H-pyrazol-4-yl]-1-[(4-methylphenyl)sulfonyl]-1H-pyrrolo[2,3-b]pyridine

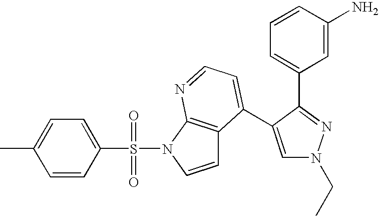

Aqueous HCl (2 N, 1 mL) was added to a suspension of 4-[1-ethyl-3-(3-nitrophenyl)-1H-pyrazol-4-yl]-1-[(4-methylphenyl)sulfonyl]-1 pyrrolo[2,3-b]pyridine (1.1 g, 2.2 mmol) and tin (1.3 g, 11.0 mmol) in EtOH (100 mL). The mixture was stirred at reflux for 4 h, and directly charged into SCX cartridge. After SCX purification, the residue was purified by Yamazen Fast Flow Liquid Chromatography on a silica gel column (EtOAc:Hexane=1:4 to 1:1) to give the desired product (780 mg, 78%). ¹H NMR (400 MHz, DMSO-d6) ppm 1.45 (t, 3H, J=7.3 Hz), 2.35 (s, 3H), 4.21 (q, 2H, J=7.3 Hz), 5.04 (brs, 2H), 6.33-6.37 (m, 1H), 6.48 (ddd, 1H, J=1.0, 2.3, 8.1 Hz), 6.59 (d, 1H, J=4.0 Hz), 6.65 (dd, 1H, J=1.8, 1.8 Hz), 6.88 (dd, 1H, J=7.8, 8.1 Hz), 7.00 (d, 1H, J=5.1 Hz), 7.43 (d, 2H, J=8.1 Hz), 7.83 (d, 1H, J=4.0 Hz), 8.00 (d, 2H, J=8.1 Hz), 8.16 (s, 1H), 8.21 (d, 1H, J=5.1 Hz). MS (ESI): m/z 458 (M+1)+

### Step B

### 3-[1-Ethyl-4-(1H-pyrrolo[2,3-b]pyridin-4-yl)-1H-pyrazol-3-yl]aniline

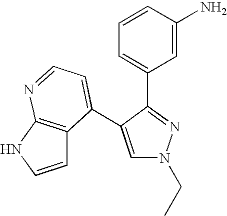

To a solution of 4-[1-ethyl-3-(3-nitrophenyl)-1H-pyrazol-4-yl]-1-[(4-methylphenyl)sulfonyl]-1H pyrrolo[2,3-b]pyridine (778 mg, 1.7 mmol) in MeOH (50 mL) was added 6N aqueous NaOH (0.5 mL). After stirring the resulting mixture at 40° C. for 1 h, the reaction was quenched by saturated aqueous NH₄Cl, and extracted with CH₂Cl₂ (20 mL×3 times). The organic layer was washed with brine, dried over Na₂SO₄, and then evaporated to dryness under reduced pressure. The residue was purified by silica gel chromatography (EtOAc:Hex=1:4 to 1:1), and the corresponding product was obtained as a white solid (511.9 mg). ¹H NMR (400 MHz, DMSO-d6) ppm 1.47 (t, 3H, J=7.3 Hz), 4.22 (q, 2H, J=7.3 Hz), 5.00 (brs, 2H), 6.30 (dd, 1H, J=2.0, 3.3 Hz), 6.39-6.44 (m, 1H), 6.46 (ddd, 1H, J=0.8, 2.3, 8.1 Hz), 6.72 (dd, 1H, J=2.0, 2.0 Hz), 6.79 (d, 1H, J=4.8 Hz), 6.88 (dd, 1H, J=7.8, 8.1 Hz), 7.38 (dd, 1H, J=2.0, 3.3 Hz), 8.06 (d, 1H, J=4.8 Hz), 8.12 (s, 1H), 11.57 (brs, 1H). MS (ESI): m/z 304 (M+1)+

### General Intermediate 6

### 3-[4-(3-Bromo-1H-pyrrolo[2,3-b]pyridin-4-yl)-1-ethyl-1H-pyrazol-3-yl]aniline

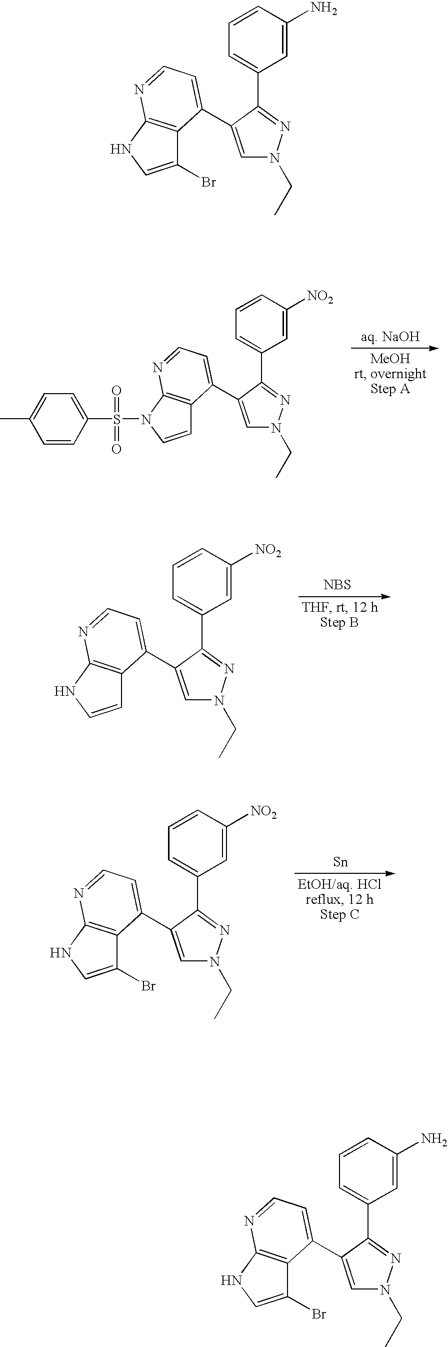

### Step A

### 4-[1-Ethyl-3-(3-nitrophenyl)-1H-pyrazol-4-yl]-1H-pyrrolo[2,3-b]pyridine

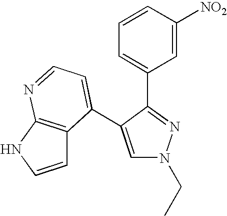

To a solution of 4-[1-ethyl-3-(3-nitrophenyl)-1H-pyrazol-4-yl]-1-[(4-methylphenyl)sulfonyl]-1H pyrrolo[2,3-b]pyridine (general intermediate 4, 350 mg, 0.7 mmol) in a mixture of DMF (2 mL), CH₂Cl₂ (30 mL), and MeOH (30 mL), was added 6 N aqueous NaOH (3 mL). After being stirred at rt overnight, the reaction was evaporated to dryness in vacuo. The resulting residue was diluted with saturated aqueous NH₄Cl, and extracted with CH₂Cl₂ (20 mL×3 times). The organic layer was washed with brine, dried over Na₂SO₄, and then evaporated to dryness under reduced pressure to give the corresponding compound (212.8 mg, 89%). ¹H NMR (400 MHz, DMSO-d6) ppm 1.51 (t, 3H, J=7.3 Hz), 4.31 (q, 2H, J=7.3 Hz), 6.15 (dd, 1H, J=2.0, 3.5 Hz), 6.88 (d, 1H, J=4.8 Hz), 7.38-7.41 (m, 1H), 7.56 (dd, 1H, J=8.1, 8.1 Hz), 7.72-7.76 (m, 1H), 8.12 (ddd, 1H, J=1.0, 2.5, 8.1 Hz), 8.15 (d, 1H, J=4.8 Hz), 8.22-8.24 (m, 1H), 8.25 (s, 1H), 11.70 (brs, 1H). MS (ESI): m/z 334 (M+1)+

### Step B

### 3-Bromo-4-[1-ethyl-3-(3-nitrophenyl)-1H-pyrazol-4-yl]-1H-pyrrolo[2,3-b]pyridine

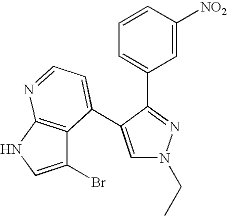

NBS (26.4 mg, 0.15 mmol) was added to a solution of 4-[1-ethyl-3-(3-nitrophenyl)-1H-pyrazol-4-yl]-1H-pyrrolo[2,3-b]pyridine (45 mg, 0.13 mmol) in THF (5 mL). After being stirred at rt overnight, the reaction was quenched by water, and extracted with CH₂Cl₂ (20 mL×3 times). The organic layer was washed with brine, dried over Na₂SO₄, and then evaporated to dryness under reduced pressure to give the corresponding compound, which was used in the next step without further purification.

### Step C

### 3-[4-(3-Bromo-1H-pyrrolo[2,3-b]pyridin-4-yl)-1-ethyl-1H-pyrazol-3-yl]aniline

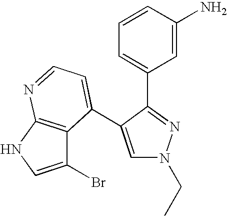

To a suspension of the residue obtained in Step B and tin (77.1 mg, 0.65 mmol) in EtOH (10 mL), was added 6N aqueous HCl (1 mL). After refluxing overnight, the reaction mixture was directly charged into SCX cartridge. After SCX purification, the residue was recrystallized with CH₂Cl₂ and MeOH to give the corresponding compound. ¹H NMR (400 MHz, DMSO-d6) ppm 1.45 (t, 3H, J=7.3 Hz), 4.21 (q, 2H, J=7.3 Hz), 4.94 (brs, 2H), 6.25-6.28 (m, 1H), 6.37 (ddd, 1H, J=0.8, 2.0, 7.8 Hz), 6.74 (dd, 1H, J=7.8, 7.8 Hz), 6.81 (dd, 1H, J=2.0, 2.0 Hz), 6.85 (d, 1H, J=4.8 Hz), 7.62 (d, 1H, J=2.8 Hz), 7.85 (s, 1H), 8.19 (d, 1H, J=4.8 Hz), 12.10 (d, 1H, J=2.0 Hz). MS (ESI): m/z 382, 384 (M+1)+

### General Intermediate 7

### 3-[4-(3-Chloro-1H-pyrrolo[2,3-b]pyridin-4-yl)-1-ethyl-1H-pyrazol-3-yl]aniline

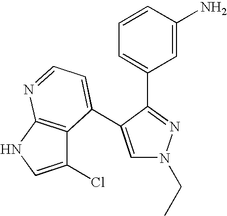

A similar procedure as general intermediate 6 was used, with NCS substituting for NBS, to prepare the title intermediate. ¹H NMR (400 MHz, DMSO-d6) ppm 1.45 (t, 3H, J=7.3 Hz), 4.21 (q, 2H, J=7.3 Hz), 4.95 (brs, 2H), 6.27-6.30 (m, 1H), 6.38 (ddd, 1H, J=8.1, 2.5, 1.0 Hz), 6.76 (dd, 1H, J=7.8, 7.8 Hz), 6.79 (dd, 1H, J=1.9, 1.9 Hz), 6.83 (d, 1H, J=4.8 Hz), 7.58 (d, 1H, J=1.9 Hz), 7.87 (s, 1H), 8.18 (d, 1H, J=4.8 Hz), 12.00 (brs, 1H).

MS (ESI): m/z 338 (M+1)+

### General Intermediate 8: Ethyl 4-(4,4,5,5-tetramethyl-1,3,2-dioxaborolan-2-yl)-1H-pyrrolo[2,3-b]pyridine-2-carboxylate

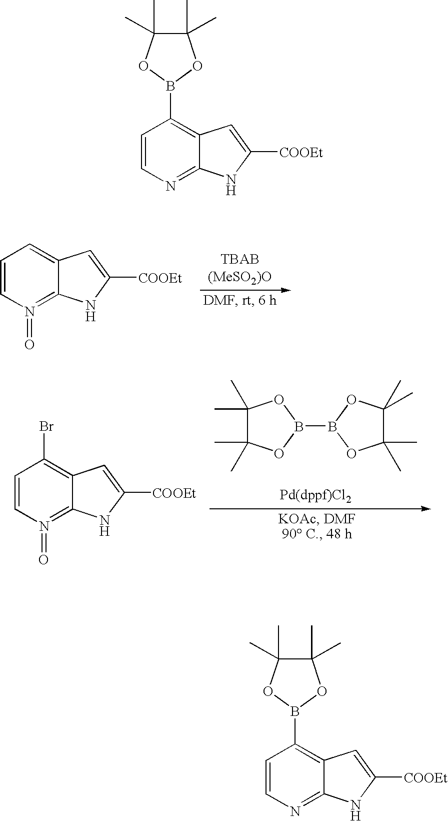

### Step A: Ethyl 4-bromo-1H-pyrrolo[2,3-b]pyridine-2-carboxylate

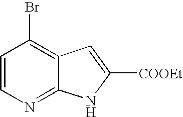

Ethyl 1H-pyrrolo[2,3-b]pyridine-2-carboxylate 7-oxide (1.0 g, 5.0 mmol), which was prepared according to WO2000044753, was added to a suspension of tetramethylammonium bromide (1.2 g, 7.5 mmol) in DMF (50 mL). The resulting mixture was cooled to 0° C. and methanesulfonic anhydride (1.7 g, 10 mmol) was added portion wise. After being warmed up to rt and stirred for another 6 h, the reaction mixture was poured into water (100 mL). By neutralizing it with 50% aqueous sodium hydroxide, the resulting solution was extracted with AcOEt, followed by washing the organic layer with brine and water. Concentration in vacuo gave the residue, which underwent SCX purification to afford the desired compound as a pale yellow solid (1.0 g, 77%). ¹H NMR (400 MHz, DMSO-d6) ppm 1.35 (t, 3H, J=7.1 Hz), 4.36 (q, 2H, J=7.1 Hz), 7.05 (d, 1H, J=2.0 Hz), 7.48 (d, 1H, J=5.1 Hz), 8.28 (d, 1H, J=5.1 Hz), 12.95 (brs, 1H). MS (ESI): m/z 269, 271 (M+1)+

### Step B: Ethyl 4-(4,4,5,5-tetramethyl-1,3,2-dioxaborolan-2-yl)-1H-pyrrolo[2,3-b]pyridine-2-carboxylate

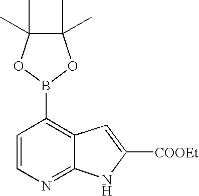

A mixture of ethyl 4-bromo-1H-pyrrolo[2,3-b]pyridine-2-carboxylate (1.0 g, 3.9 mmol), 4,4,4′,4′,5,5,5′,5′-octamethyl-2,2′-bi-1,3,2-dioxaborolane (2.1 g, 7.8 mmol), KOAc (1.2 g, 11.7 mmol) and Pd(dppf)Cl₂ (0.3 g, 0.4 mmol) in DMF (20 mL) was heated at 90° C. for 48 h under inert atmosphere. After cooling to rt, the reaction was quenched by aqueous NH₄Cl. The resulting mixture was extracted with AcOEt. The organic layer was washed with brine, water, and dried over sodium sulfate. Concentration in vacuo gave the residue, which was purified by YAMAZEN Fast Flow Liquid Chromatography (silica gel, EtOAc:hexane=1:1) to give the desired product as a white solid (0.8 g, 68%). ¹H NMR (400 MHz, DMSO-d6) ppm 1.33-1.37 (m, 15H), 4.37 (q, 2H, J=7.1 Hz), 7.29 (d, 1H, J=2.0 Hz), 7.40 (d, 1H, J=4.5 Hz), 8.44 (d, 1H, J=4.3 Hz), 12.55 (brs, 1H). MS (ESI): m/z 315 (M−1)−, 317 (M+1)+

### General Intermediate 9: 1-Ethyl-3-(3-nitrophenyl)-4-(4,4,5,5-tetramethyl-1,3,2-dioxaborolan-2-yl)-1H-pyrazole

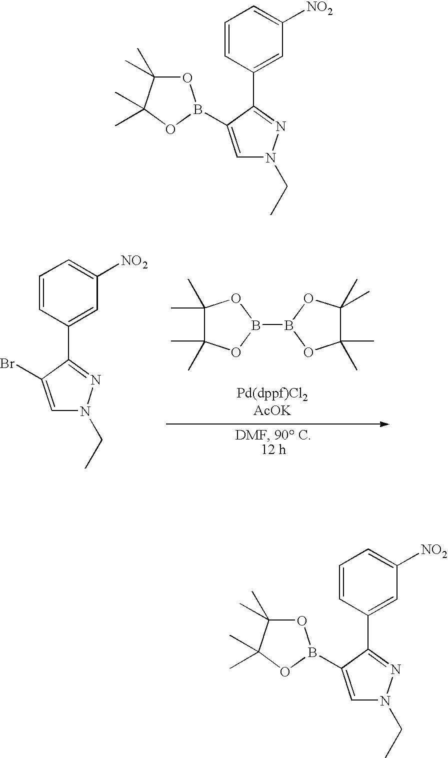

A mixture of 4-bromo-1-ethyl-3-(3-nitrophenyl)-1H-pyrazole (2.5 g, 8.6 mmol), 4,4,4′,4′,5,5,5′,5′-octamethyl-2,2′-bi-1,3,2-dioxaborolane (4.3 g, 17.1 mmol), KOAc (2.5 g, 25.7 mmol) and Pd(dppf)Cl₂ (0.6 g, 0.9 mmol) in DMF (150 mL) was heated at 90° C. overnight under inert atmosphere. After cooling to rt, 1 N aqueous NaOH was added till the aqueous layer was taken to pH 10. The aqueous layer was washed with CH₂Cl₂, carefully acidified to pH 4 with 1 N aqueous HCl, and extracted with CH₂Cl₂ (20 mL×3 times). The organic layer was dried with Na₂SO₄, and concentrated under reduced pressure to give the desired boronic ester as a brown solid. ¹H NMR (400 MHz, DMSO-d6) ppm 1.30 (s, 12H), 1.42 (t, J=7.20 Hz, 3H), 4.22 (q, J=7.33 Hz, 2H), 7.68 (dd, J=7.8, 7.8 Hz, 1H), 8.11 (s, 1H), 8.18 (ddd, J=1.0, 2.4, 7.8 Hz, 1H), 8.33 (ddd, J=1.0, 1.5, 7.8 Hz, 1H), 8.94 (dd, J=1.5, 2.4 Hz, 2H). MS (ESI): m/z 344 (M+1)+

### General Intermediate 10

### Ethyl 4-[3-(3-aminophenyl)-1-ethyl-1H-pyrazol-4-yl]-1H-pyrrolo[2,3-b]pyridine-2-carboxylate

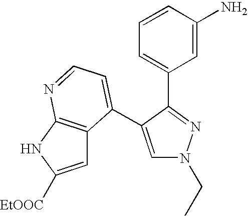

### Procedure 1:

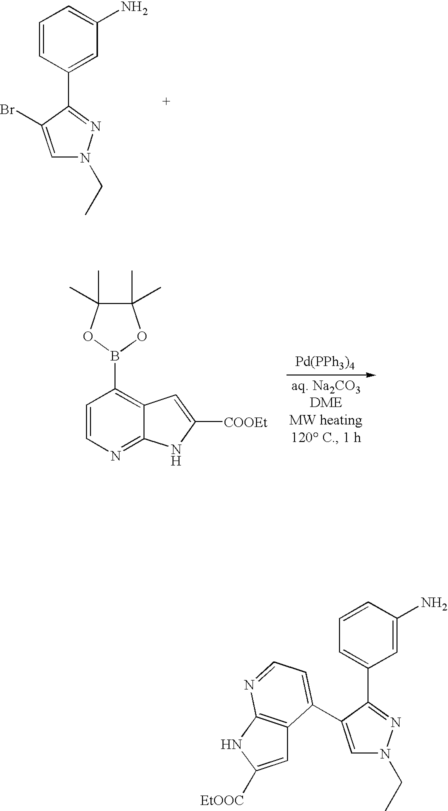

3-(4-Bromo-1-ethyl-1H-pyrazol-3-yl)aniline (general intermediate 2, 470 mg, 1.8 mmol) and ethyl 4-(4,4,5,5-tetramethyl-1,3,2-dioxaborolan-2-yl)-1H-pyrrolo[2,3-b]pyridine-2-carboxylate (general intermediate 8, 559 mg, 1.8 mmol) were dissolved in DME (18 mL) and aqueous Na₂CO₃ (2 M, 0.5 mL). The resulting solution and Pd(PPh₃)₄ (204.5 mg, 0.18 mmol) were added to a microwave vial. After capping, the mixture was heated with Creator at 120° C. for 60 min. The precipitate was removed by filtration and rinsed with CH₂Cl₂. After SCX purification, the residue was purified by Yamazen Fast Flow Liquid Chromatography on a silica gel column (EtOAc:Hexane=1:1 to 1:0) to afford the desired product (113.7 mg, 17%). ¹H NMR (400 MHz, DMSO-d6) ppm 1.32 (t, 3H, J=7.3 Hz), 1.48 (t, 3H, J=7.3 Hz), 4.25 (q, 2H, J=7.3 Hz), 4.32 (q, 2H, J=7.3 Hz), 5.04 (brs, 2H), 6.39-6.43 (m, 1H), 6.48 (ddd, 1H, J=1.0, 1.8, 7.8 Hz), 6.70 (dd, 1H, J=1.8, 1.8 Hz), 6.86 (d, 1H, J=5.1 Hz), 6.90 (dd, 1H, J=7.8, 7.8 Hz), 7.04 (d, 1H, J=1.8 Hz), 8.24-8.26 (m, 2H), 12.46 (brs, 1H). MS (ESI): m/z 376 (M+1)+

### Procedure 2:

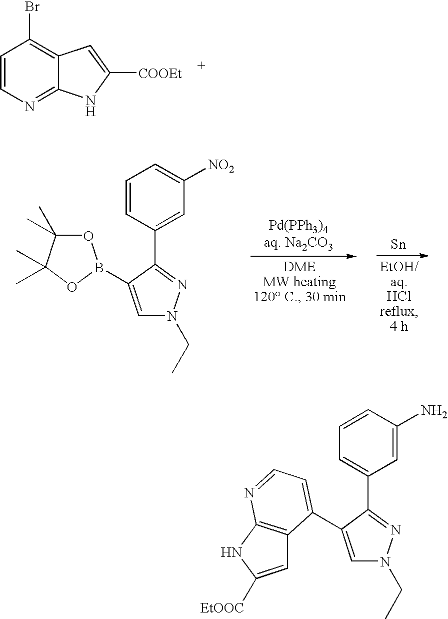

### Step A: Ethyl 4-[1-ethyl-3-(3-nitrophenyl)-1H-pyrazol-4-yl]-1H-pyrrolo[2,3-b]pyridine-2-carboxylate

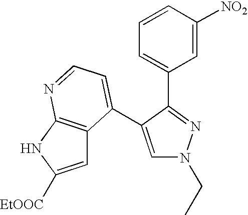

Ethyl 4-bromo-1H-pyrrolo[2,3-b]pyridine-2-carboxylate (step A in general intermediate 8, 134.6 mg, 0.5 mmol) and 1-ethyl-3-(3-nitrophenyl)-4-(4,4,5,5-tetramethyl-1,3,2-dioxaborolan-2-yl)-1H-pyrazole (general intermediate 9, 171.5 mg, 0.5 mmol) were dissolved in DME (4 mL) and aqueous Na₂CO₃ (2 M, 0.5 mL). The resulting solution and Pd(PPh₃)₄ (34.7 mg, 0.03 mmol) were added to a microwave vial. After capping, the mixture was heated with Creator at 120° C. for 30 minutes. SCX purification afforded the residue, which was directly used for the next step without further purification.

### Step B: Ethyl 4-[3-(3-aminophenyl)-1-ethyl-1H-pyrazol-4-yl]-1H-pyrrolo[2,3-b]pyridine-2-carboxylate

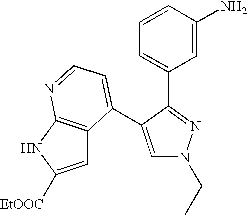

To a suspension of the above mixture and tin (296.8 mg, 2.5 mmol) in EtOH (30 mL) was added 6N aqueous HCl (1 mL). After being refluxed for 4 h, the mixture underwent SCX purification. The residue was purified by Yamazen Fast Flow Liquid Chromatography on silica gel column (EtOAc:Hexane=1:1 to 1:0) to afford the desired product (32.1 mg, 17%).

### Example 1

### N-{3-[1-Ethyl-4-(1H-pyrrolo[2,3-b]pyridin-4-yl)-1H-pyrazol-3-yl]phenyl}-N-phenylurea

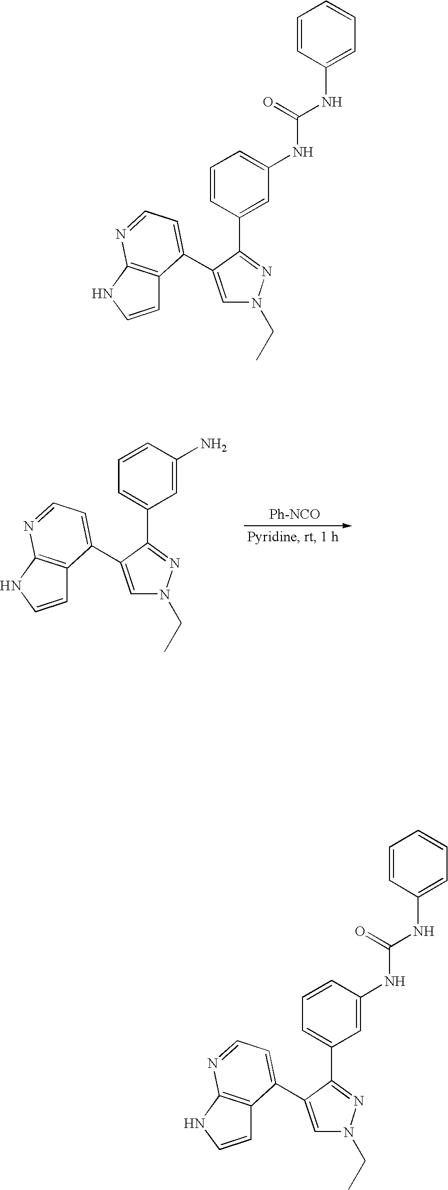

Phenyl isocyanate (7.1 mg, 0.06 mmol) was added to a solution of 3-[1-ethyl-4-(1H-pyrrolo[2,3-b]pyridin-4-yl)-1H-pyrazol-3-yl]aniline (general intermediate 5, 15 mg, 0.05 mmol) in pyridine (1 mL), and the reaction mixture was stirred at rt for 1 h. After removing the solvent in vacuo, the residue was purified by LC/MS to give the title compound. ¹H NMR (400 MHz, DMSO-d6) ppm 1.50 (t, 3H, J=7.2 Hz), 4.27 (q, 2H, J=7.2 Hz), 6.27 (dd, 1H, J=2.0, 3.5 Hz), 6.81 (d, 1H, J=5.1 Hz), 6.89 (ddd, 1H, J=1.1, 1.3, 7.6 Hz), 6.95 (d, 1H, J=7.2 Hz), 7.16 (dd, 1H, J=7.8, 7.8 Hz), 7.23-7.31 (m, 2H), 7.38-7.51 (m, 5H), 8.10 (d, 1H, J=5.1 Hz), 8.18 (s, 1H), 8.56 (s, 1H), 8.64 (s, 1H), 11.62 (brs, 1H). MS (ESI): m/z 423 (M+1)+

### Example 2

### N-(3-Chlorophenyl)-N-{3-[1-ethyl-4-(1H-pyrrolo[2,3-b]pyridin-4-yl)-1H-pyrazol-3-yl]phenyl}urea

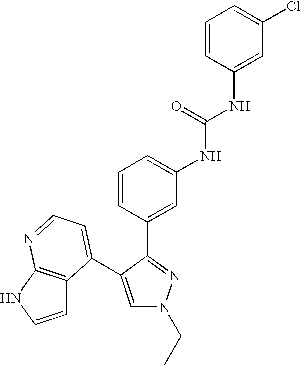

A similar procedure as example 1 was used, with 3-chlorophenyl isocyanate substituting for phenyl isocyanate, to prepare the title compound. ¹H NMR (400 MHz, DMSO-d6) ppm 1.50 (t, 3H, J=7.2 Hz), 4.27 (q, 2H, J=7.2 Hz), 6.26 (dd, 1H, J=2.0, 3.5 Hz), 6.81 (d, 1H, J=5.1 Hz), 6.89-6.93 (m, 1H), 7.01 (ddd, 1H, J=1.0, 2.0, 8.0 Hz), 7.17 (dd, 1H, J=8.0, 8.0 Hz), 7.23 (ddd, 1H, J=1.0, 2.0, 8.0 Hz), 7.28 (dd, 1H, J=8.0, 8.0 Hz), 7.39 (dd, 1H, J=2.0, 3.5 Hz), 7.46 (ddd, 1H, J=1.0, 2.0, 8.0 Hz), 7.51 (dd, 1H, J=2.0, 2.0 Hz), 7.68 (dd, 1H, J=2.0, 2.0 Hz), 8.10 (d, 1H, J=5.1 Hz), 8.18 (s, 1H), 8.74 (s, 1H), 8.78 (s, 1H), 11.62 (brs, 1H). MS (ESI): m/z 457 (M+1)+

### Example 3

### N-{3-[1-Ethyl-4-(1H-pyrrolo[2,3-b]pyridin-4-yl)-1H-pyrazol-3-yl]phenyl}-N-[2-fluoro-5-(trifluoromethyl)phenyl]urea

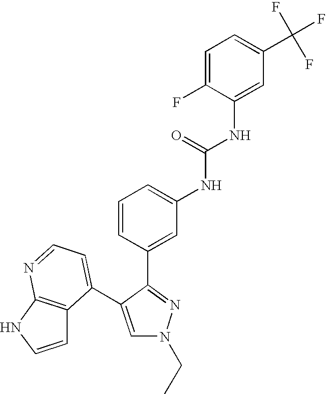

A similar procedure as example 1 was used, with 2-fluoro-5-(trifluoromethyl)phenyl isocyanate substituting for phenyl isocyanate, to prepare the title compound. ¹H NMR (400 MHz, DMSO-d6) ppm 1.50 (t, 3H, J=7.3 Hz), 4.27 (q, 2H, J=7.3 Hz), 6.25 (dd, 1H, J=1.8, 3.5 Hz), 6.82 (d, 1H, J=5.1 Hz), 6.94 (ddd, 1H, J=1.1, 1.3, 7.3 Hz), 7.19 (dd, 1H, J=8.0, 8.0 Hz), 7.35-7.41 (m, 2H), 7.45-7.52 (m, 3H), 8.10 (d, 1H, J=5.1 Hz), 8.19 (s, 1H), 8.59 (dd, 1H, J=2.3, 7.3 Hz), 8.81 (s, 1H), 9.15 (s, 1H), 11.63 (brs, 1H). MS (ESI): m/z 509 (M+1)+

### Example 4

### N-[4-Chloro-3-(trifluoromethyl)phenyl]-N-{3-[1-ethyl-4-(1H-pyrrolo[2,3-b]pyridin-4-yl)-1H-pyrazol-3-yl]phenyl}urea

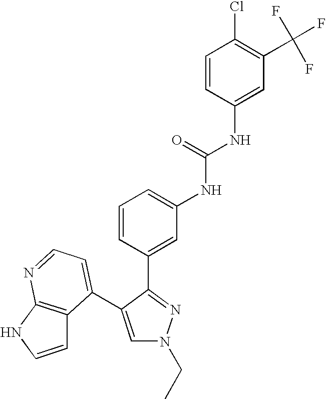

A similar procedure as example 1 was used, with 4-chloro-3-(trifluoromethyl)phenyl isocyanate substituting for phenyl isocyanate, to prepare the title compound. ¹H NMR (400 MHz, DMSO-d6) ppm 1.50 (t, 1H, J=7.3 Hz), 4.27 (q, 1H, J=7.3 Hz), 6.26 (dd, 1H, J=1.8, 3.3 Hz), 6.81 (d, 1H, J=4.8 Hz), 6.92 (ddd, 1H, J=1.3, 1.3, 7.8 Hz), 7.17 (d, 1H, J=7.8, 7.8 Hz), 7.39 (dd, 1H, J=2.5, 3.3 Hz), 7.46 (ddd, 1H, J=1.3, 1.8, 7.8 Hz), 7.53 (dd, 1H, J=1.8, 1.8 Hz), 7.59-7.61 (m, 2H), 8.07-8.09 (m, 1H), 8.10 (d, 1H, J=4.8 Hz), 8.18 (s, 1H), 8.85 (s, 1H), 9.08 (s, 1H), 11.62 (brs, 1H). MS (ESI): m/z 525 (M+1)+

### Example 5

### N-{3-[1-Ethyl-4-(1H-pyrrolo[2,3-b]pyridin-4-yl)-1H-pyrazol-3-yl]phenyl}-N-[3-(methyloxy)phenyl]urea

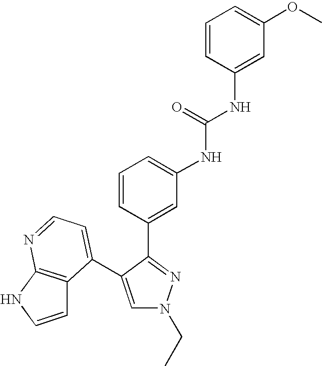

A similar procedure as example 1 was used, with 3-methoxyphenyl isocyanate substituting for phenyl isocyanate, to prepare the title compound. ¹H NMR (400 MHz, DMSO-d6) ppm 1.49 (t, 3H, J=7.2 Hz), 3.72 (s, 3H), 4.26 (q, 2H, J=7.2 Hz), 6.26 (dd, 1H, J=1.8, 3.5 Hz), 6.54 (ddd, 1H, J=0.8, 2.3, 8.1 Hz), 6.81 (d, 1H, J=5.1 Hz), 6.86-6.92 (m, 2H), 7.12-7.19 (m, 3H), 7.39 (dd, 1H, J=2.8, 3.5 Hz), 7.45 (ddd, 1H, J=0.8, 2.5, 8.1 Hz), 7.49 (dd, 1H, J=1.8, 1.8 Hz), 8.09 (d, 1H, J=5.1 Hz), 8.18 (s, 1H), 8.57 (s, 1H), 8.63 (s, 1H), 11.62 (brs, 1H). MS (ESI): m/z 453 (M+1)+

### Example 6

### N-(2,6-Difluorophenyl)-N-{3-[1-ethyl-4-(1H-pyrrolo[2,3-b]pyridin-4-yl)-1H-pyrazol-3-yl]phenyl}urea

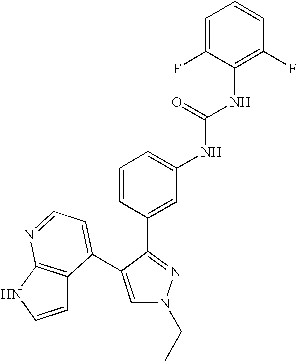

A similar procedure as example 1 was used, with 2,6-difluorophenyl isocyanate substituting for phenyl isocyanate, to prepare the title compound. ¹H NMR (400 MHz, DMSO-d6) ppm 1.48 (t, 3H, J=7.3 Hz), 4.25 (q, 2H, J=7.3 Hz), 6.26 (dd, 1H, J=1.8, 3.5 Hz), 6.80 (d, 1H, J=5.1 Hz), 6.85 (ddd, 1H, J=0.8, 1.8, 8.1 Hz), 7.09-7.18 (m, 3H), 7.26-7.34 (m, 1H), 7.39 (dd, 1H, J=2.8, 3.5 Hz), 7.45 (ddd, 1H, J=0.8, 2.0, 8.1 Hz), 7.55 (d, 1H, J=1.8, 1.8 Hz), 8.02 (s, 1H), 8.09 (d, 1H, J=5.1 Hz), 8.17 (s, 1H), 8.94 (s, 1H), 11.61 (brs, 1H).

MS (ESI): m/z 459 (M+1)+

### Example 7

### N-{3-[1-Ethyl-4-(1H-pyrrolo[2,3-b]pyridin-4-yl)-1H-pyrazol-3-yl]phenyl}-N-{4-[(phenylmethyl)oxy]phenyl}urea

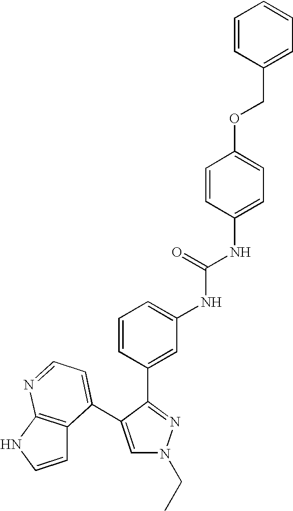

A similar procedure as example 1 was used, with 4-benzyloxyphenyl isocyanate substituting for phenyl isocyanate, to prepare the title compound. ¹H NMR (400 MHz, DMSO-d6) ppm 1.49 (t, 3H, J=7.3 Hz), 4.26 (q, 2H, J=7.3 Hz), 5.05 (s, 2H), 6.27 (dd, 1H, J=2.0, 3.5 Hz), 6.81 (d, 1H, J=5.1 Hz), 6.87 (ddd, 1H, J=1.3, 1.5, 7.8 Hz), 6.93 (d, 1H, J=9.1 Hz), 7.14 (dd, 1H, J=7.8, 7.8 Hz), 7.28-7.50 (m, 10H), 8.09 (d, 1H, J=5.1 Hz), 8.18 (s, 1H), 8.41 (s, 1H), 8.60 (s, 1H), 11.62 (brs, 1H). MS (ESI): m/z 529 (M+1)+

### Example 8

### N-Cyclohexyl-N-{3-[1-ethyl-4-(1H-pyrrolo[2,3-b]pyridin-4-yl)-1H-pyrazol-3-yl]phenyl}urea

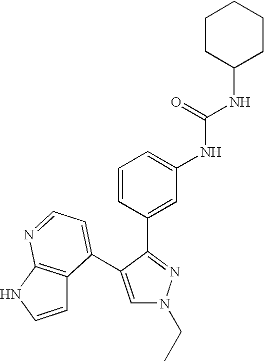

A similar procedure as example 1 was used, with isocyanatocyclohexane substituting for phenyl isocyanate, to prepare the title compound. ¹H NMR (400 MHz, DMSO-d6) ppm 1.07-1.21 (m, 3H), 1.22-1.32 (m, 2H), 1.45-1.56 (m, 4H), 1.57-1.68 (m, 2H), 1.72-1.82 (m, 2H), 3.36-3.47 (m, 1H), 4.25 (q, 2H, J=7.3 Hz), 5.96 (d, 1H, J=7.8 Hz), 6.26 (d, 1H, J=2.8 Hz), 6.78 (d, 1H, J=4.8 Hz), 6.78-6.82 (m, 1H), 7.08 (dd, 1H, J=7.8, 8.8 Hz), 7.37-7.42 (m, 3H), 8.08 (d, 1H, J=4.8 Hz), 8.16 (s, 1H), 8.26 (s, 1H), 11.61 (brs, 1H). MS (ESI): m/z 429 (M+1)+

### Example 9

### N-{3-[1-Ethyl-4-(1H-pyrrolo[2,3-b]pyridin-4-yl)-1H-pyrazol-3-yl]phenyl}-N-(phenylmethyl)urea

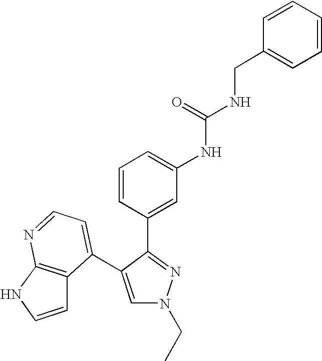

A similar procedure as example 1 was used, with benzyl isocyanate substituting for phenyl isocyanate, to prepare the title compound. ¹H NMR (400 MHz, DMSO-d6) ppm 1.48 (t, 3H, J=7.3 Hz), 4.21-4.29 (m, 4H), 6.26 (dd, 1H, J=2.0, 3.5 Hz), 6.53 (dd, 1H, J=6.1, 6.1 Hz), 6.76-6.82 (m, 2H), 7.09 (dd, 1H, J=7.8, 8.1 Hz), 7.20-7.35 (m, 5H), 7.39 (dd, 1H, J=2.5, 3.3 Hz), 7.42 (ddd, 1H, J=1.0, 2.0, 8.1 Hz), 7.47 (t, 1H, J=1.8, 2.0 Hz), 8.08 (d, 1H, J=4.8 Hz), 8.16 (s, 1H), 8.55 (s, 1H), 11.61 (brs, 1H). MS (ESI): m/z 437 (M+1)+

### Example 10

### N-{3-[1-Ethyl-4-(1H-pyrrolo[2,3-b]pyridin-4-yl)-1H-pyrazol-3-yl]phenyl}-N-[4-(trifluoromethyl)phenyl]urea

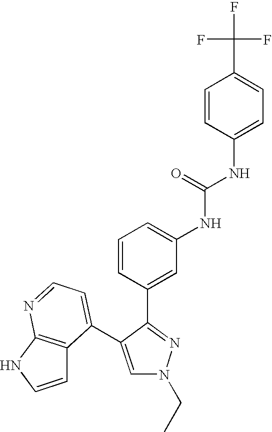

A similar procedure as example 1 was used, with 4-trifluorophenyl isocyanate substituting for phenyl isocyanate, to prepare the title compound. ¹H NMR (400 MHz, DMSO-d6) ppm 1.49 (t, 3H, J=7.3 Hz), 4.26 (q, 2H, J=7.3 Hz), 6.26 (dd, 1H, J=2.0, 3.3 Hz), 6.81 (d, 1H, J=5.1 Hz), 6.90-6.95 (m, 1H), 7.18 (dd, 1H, J=7.8, 8.0 Hz), 7.39 (dd, 1H, J=2.5, 3.3 Hz), 7.47 (ddd, 1H, J=1.0, 2.0, 8.1 Hz), 7.51-7.54 (m, 1H), 7.59-7.65 (m, 4H), 8.09 (d, 1H, J=5.1 Hz), 8.18 (s, 1H), 8.80 (s, 1H), 9.00 (s, 1H), 11.62 (brs, 1H). MS (ESI): m/z 491 (M+1)+

### Example 11

### N-(2-Chlorophenyl)-N-{3-[1-ethyl-4-(1H-pyrrolo[2,3-b]pyridin-4-yl)-1H-pyrazol-3-yl]phenyl}urea

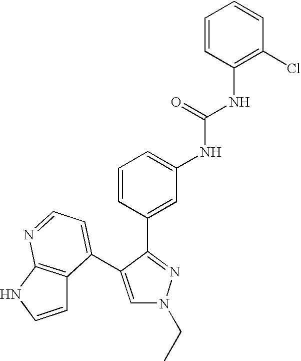

A similar procedure as example 1 was used, with 2-chlorophenyl isocyanate substituting for phenyl isocyanate, to prepare the title compound. ¹H NMR (400 MHz, DMSO-d6) ppm 1.49 (t, 3H, J=7.2 Hz), 4.26 (q, 2H, J=7.2 Hz), 6.27 (dd, 1H, J=1.5 3.3 Hz), 6.82 (d, 1H, J=5.1 Hz), 6.89-6.92 (m, 1H), 7.02 (ddd, 1H, J=1.5, 7.6, 8.1 Hz), 7.15-7.22 (m, 1H), 7.29 (ddd, 1H, J=1.5, 7.6, 8.6 Hz), 7.40 (dd, 1H, J=2.3, 3.3 Hz), 7.44 (dd, 1H, J=1.5, 8.1 Hz), 7.48-7.53 (m, 2H), 8.10 (d, 1H, J=5.1 Hz), 8.14 (dd, 1H, J=1.5, 8.1 Hz), 8.18 (s, 1H), 8.24 (s, 1H), 9.39 (s, 1H), 11.63 (brs, 1H). MS (ESI): m/z 457 (M+1)+

### Example 12

### N-{3-[1-Ethyl-4-(1H-pyrrolo[2,3-b]pyridin-4-yl)-1H-pyrazol-3-yl]phenyl}-N-phenylthiourea

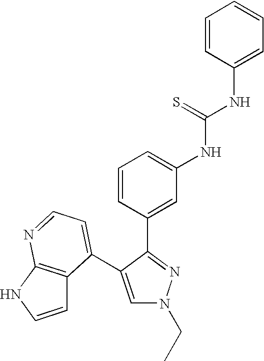

A similar procedure as example 1 was used, with phenyl thioisocyanate substituting for phenyl isocyanate, to prepare the title compound. ¹H NMR (400 MHz, DMSO-d6) ppm 1.49 (t, 3H, J=7.2 Hz), 4.25 (q, 2H, J=7.2 Hz), 6.28 (d, 1H, J=3.3 Hz), 6.82 (d, 1H, J=4.8 Hz), 7.03-7.08 (m, 1H), 7.09-7.15 (m, 1H), 7.21 (dd, 1H, J=7.8, 7.8 Hz), 7.28-7.36 (m, 2H), 7.37-7.44 (m, 3H), 7.49 (ddd, 1H, J=1.0, 2.3, 8.1 Hz), 7.55 (dd, 1H, J=1.8, 1.8 Hz), 8.08 (d, 1H, J=4.8 Hz), 8.18 (s, 1H), 9.74 (s, 1H), 9.76 (s, 1H), 11.63 (brs, 1H).

MS (ESI): m/z 439 (M+1)+

### Example 13

### N-{3-[4-(3-Chloro-1H-pyrrolo[2,3-b]pyridin-4-yl)-1-ethyl-1H-pyrazol-3-yl]phenyl}-N-phenylurea

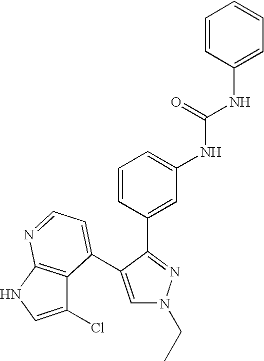

Phenyl isocyanate (6.6 mg, 0.06 mmol) was added to a solution of 3-[4-(3-chloro-1H-pyrrolo[2,3-b]pyridin-4-yl)-1-ethyl-1H-pyrazol-3-yl]aniline (general intermediate 7, 17.0 mg, 0.05 mmol) in pyridine (1 mL), and reaction mixture was stirred at rt for 1 hour. After removing the solvent in vacuo, the residue was purified by LC/MS to give the title compound. ¹H NMR (400 MHz, DMSO-d6) ppm 1.48 (t, 3H, J=7.2 Hz), 4.26 (q, 2H, J=7.2 Hz), 6.75-6.80 (m, 1H), 6.89 (d, 1H, J=5.1 Hz), 7.08 (dd, 1H, J=7.8, 8.1 Hz), 7.49-7.55 (m, 2H), 7.55-7.61 (m, 2H), 7.64-7.70 (m, 1H), 7.90-7.98 (m, 3H), 8.07 (dd, 1H, J=1.8, 1.8 Hz), 8.22 (d, 1H, J=5.1 Hz), 10.23 (s, 1H), 12.04 (brs, 1H). MS (ESI): m/z 457 (M+1)+

### Example 14

### N-{3-[4-(3-Bromo-1H-pyrrolo[2,3-b]pyridin-4-yl)-1-ethyl-1H-pyrazol-3-yl]phenyl}-N-phenylurea

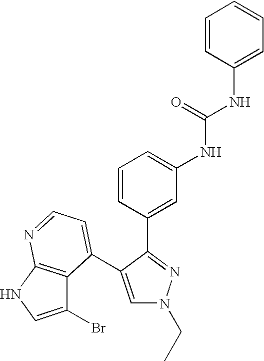

Phenyl isocyanate (6.6 mg, 0.06 mmol) was added to a solution of 3-[4-(3-bromo-1H-pyrrolo[2,3-b]pyridin-4-yl)-1-ethyl-1H-pyrazol-3-yl]aniline (general intermediate 6, 20.0 mg, 0.05 mmol) in pyridine (1 mL), and the reaction mixture was stirred at rt for 1 h. After removing the solvent in vacuo, the residue was purified by LC/MS to give the title compound. ¹H NMR (400 MHz, DMSO-d6) ppm 1.48 (t, 3H, J=7.2 Hz), 4.26 (q, 2H, J=7.2 Hz), 6.75 (ddd, 1H, J=1.0, 1.5, 8.1 Hz), 6.91 (d, 1H, J=4.8 Hz), 7.06 (dd, 1H, J=7.8, 8.1 Hz), 7.48-7.55 (m, 2H), 7.55-7.61 (m, 1H), 7.61-7.68 (m, 2H), 7.90-7.97 (m, 3H), 8.09 (dd, 1H, J=1.9, 1.9 Hz), 8.22 (d, 1H, J=4.8 Hz), 10.24 (s, 1H), 12.09 (brs, 1H). MS (ESI): m/z 501, 503 (M+1)+

### Example 15

### Ethyl 4-(1-ethyl-3-{3-[({[4-(trifluoromethyl)phenyl]amino}carbonyl)amino]phenyl}-1H-pyrazol-4-yl)-1H-pyrrolo[2,3-b]pyridine-2-carboxylate

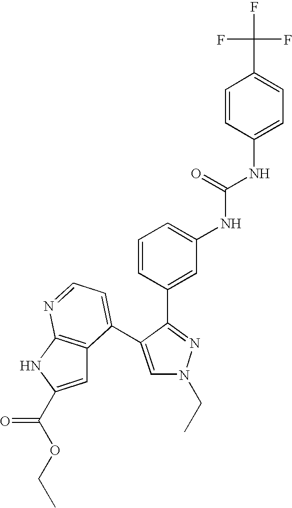

4-Trifluorophenyl isocyanate (9.3 mg, 0.05 mmol) was added to a solution of ethyl 4-[3-(3-aminophenyl)-1-ethyl-1H-pyrazol-4-yl]-1H-pyrrolo[2,3-b]pyridine-2-carboxylate (general intermediate 10, 15.0 mg, 0.04 mmol) in pyridine (1 mL), and the reaction mixture was stirred at rt for 1 h. After removing the solvent in vacuo, the residue was purified by LC/MS to give the title compound. ¹H NMR (400 MHz, DMSO-d6) ppm 1.31 (t, 3H, J=7.3 Hz), 1.50 (t, 3H, J=7.3 Hz), 4.25-4.35 (m, 4H), 6.88 (d, 1H, J=5.1 Hz), 6.91-6.95 (m, 1H), 7.00 (d, 1H, J=2.0 Hz), 7.20 (dd, 1H, J=7.8, 7.8 Hz), 7.46 (ddd, 1H, J=1.0, 2.0 7.8 Hz), 7.54 (dd, 1H, J=1.8, 2.0 Hz), 7.60-7.64 (m, 4H), 8.28 (d, 1H, J=5.1 Hz), 8.31 (s, 1H), 8.82 (s, 1H), 9.02 (s, 1H), 12.50 (brs, 1H). MS (ESI): m/z 563 (M+1)+

### Example 16

### 4-(1-Ethyl-3-{3-[({[4-(trifluoromethyl)phenyl]amino}carbonyl)amino]phenyl}-1H-pyrazol-4-yl)-1H-pyrrolo[2,3-b]pyridine-2-carboxylic Acid

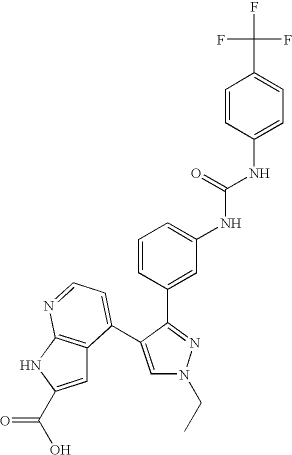

To a solution of 4-(1-ethyl-3-{3-[({[4-(trifluoromethyl)phenyl]amino}carbonyl)amino]phenyl}-1H-pyrazol-4-yl)-1H-pyrrolo[2,3-b]pyridine-2-carboxylate (10.0 mg, 0.02 mmol) in MeOH (1 mL), was added 6 N aqueous NaOH (0.1 mL). The mixture was stirred at reflux for 1 h. After removing the solvents in vacuo, the residue was purified by LC/MS to give the title compound. ¹H NMR (400 MHz, DMSO-d6) ppm 1.50 (t, 3H, J=7.3 Hz), 4.28 (q, 2H, J=7.3 Hz), 6.56 (s, 1H), 6.76 (d, 1H, J=4.8 Hz), 7.06-7.12 (m, 1H), 7.16-7.25 (m, 2H), 7.53-7.60 (m, 2H), 7.74-7.82 (m, 3H), 8.16 (d, 1H, J=5.1 Hz), 8.22 (s, 1H), 10.58 (brs, 1H), 11.07 (brs, 1H). MS (ESI): m/z 535 (M+1)+

Compounds of the present invention were tested for B-Raf family protein tyrosine kinase inhibitory activity in enzyme assays and cell proliferation assays.

### A. Enzyme Assay:

Compounds of the present invention were tested against B-Raf in a fluorescence anisotropy binding assay. In general, the enzyme, fluorescent ligand, and test compound were allowed to come to equilibrium under conditions where there is a significant difference in the observed anisotropy, reflective of binding of the ligand to the enzyme, in the presence (>10×Kᵢ) or absence of test compound. The assay conditions were set so that the enzyme concentration is ≧1×K_{f} and the ligand concentration is less than the enzyme concentration.

Test compounds were serially diluted in DMSO and 0.1 μL was plated in low volume black 384-well plates. The assay was initiated by the addition of 10 μL of an enzyme/ligand mix with a final assay composition of 50 mM HEPES (pH 7.3), 10 mM MgCl2, 1 mM CHAPS, 1 mM DTT, 1 nM fluorescent ligand, 2 nM competent B-Raf (competency determined as fraction of enzyme able to bind fluorescent ligand), and 0.169 nM-10 μM test compound. After incubation for two hours, the fluorescence anisotropy was read on a LJL Acquest with excitation at 485 nM and emission at 530 nM. Recombinant, His-tagged B-Raf (residues 462-770) that had been expressed in baculovirus was used for these experiments.

The data for dose responses were plotted as % Inhibition versus compound concentration following normalization using the formula 100*((U−C1)/(C₂−C₁)) where U is the unknown value, C1 is the average control value obtained for 1% DMSO, and C2 is the average control value for a known inhibitor. Curve fitting was performed with the equation y=A+((B−A)/(1+(10^{X}/10^{C})^{D})) where A is the y minimum, B is the y maximum, C is the log(XC₅₀), and D is the Hill slope. The results for each compound were recorded as pIC₅₀ values (−C in the above equation).

### DEFINITIONS

Kᵢ=dissociation constant for inhibitor binding
K_{f}=dissociation constant for fluorescent ligand binding

The fluorescent ligand is the following compound:

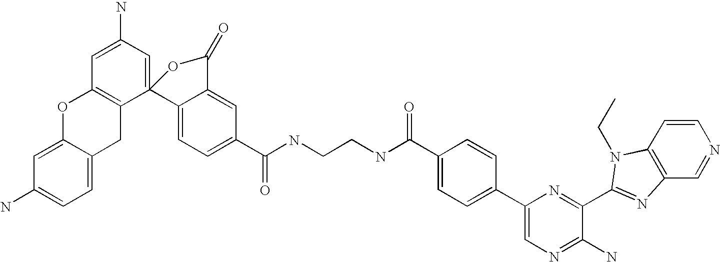

The exemplified compounds were run in the recited assay and each resulted in a measured pIC50 greater than 6.0 against B-RAF.

### B. Cellular Assays:

B-Raf mediated phosphorylation of MEK1 was measured in a cellular assay. Expression constructs for B-Raf and FLAG-tagged MEK1 (a B-raf substrate) were co-transfected in 3T3 cells and gene expression was induced using the GeneSwitch™ system for inducible mammalian expression (Invitrogen). Four hours following the induction of expression of B-Raf and MEK1, cells were exposed to the test compounds for two hours. The cells were then lysed, and then an immunoassay was performed using anti-phospho-MEK1/2 (Cell Signaling Technlogoies) to detect the percent inhibition of MEK1 phosphorylation. The concentration of the test compound that inhibited 50% of MEK1 phosphorylation (IC₅₀) was interpolated using nonlinear regression (Levenberg-Marquardt) and the equation y=1+((b−a)/(1+(10×/10c)d), where c is equal to the IC50. The results were as follows:

| Examples | Potency |
|---|---|
| 16 | Not tested |
| 7 and 13 | + |
| 1-6, 8-12, and 14-15 | ++ |
| 1-5, 10, 11, and 15 | +++ |

+ is > 6.0 μM
++ is ≦ 6.0 μM
+++ is ≦ 1.0 μM

What is claimed is:

1. A compound of formula (I): 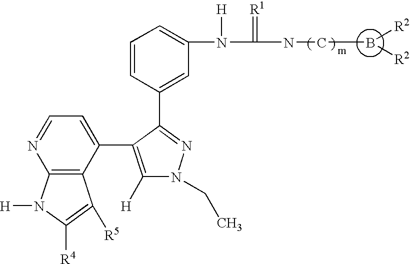
and pharmaceutically acceptable salts and solvates thereof wherein:
R¹ is selected from O and S;
m is 0 or 1;
B is a 6 membered cycloalkyl or aryl ring;
R² and R³ are independently selected from H, alkoxy, haloalkyl, halo, and phenalkoxy;
R⁴ is selected from H, —C(O)OH, and —C(O)—O—CH₂—CH₃; and
R⁵ is selected from H and halo.

2. The compound of claim 1, wherein R¹ is O.

3. The compound of claim 1, wherein R¹ is S.

4. The compound of claim 1, wherein at least one of R² and R³ is selected from alkoxy, haloalkyl, halo, and phenalkoxy.

5. The compound of claim 1, wherein at least one of R² and R³ is trifluoroalkyl.

6. The compound of claim 5, wherein at least one of R² and R³ is trifluoromethyl.

7. The compound of claim 1, wherein at least one of R² and R³ is halo.

8. The compound of claim 1, wherein both R² and R³ are H.

9. The compound of claim 1, wherein both R⁴ and R⁵ are H.

10. The compound of claim 1, wherein R⁴ is —C(O)OH.

11. The compound of claim 1, wherein R⁴ is —C(O)—O—CH₂—CH₃.

12. The compound of claim 1, wherein R⁵ is halo.

13. A compound selected from:
N-{3-[1-Ethyl-4-(1H-pyrrolo[2,3-b]pyridin-4-yl)-1H-pyrazol-3-yl]phenyl}-N-phenylurea;
N-(3-Chlorophenyl)-N-{3-[1-ethyl-4-(1H-pyrrolo[2,3-b]pyridin-4-yl)-1H-pyrazol-3-yl]phenyl}urea;
N-{3-[1-Ethyl-4-(1H-pyrrolo[2,3-b]pyridin-4-yl)-1H-pyrazol-3-yl]phenyl}-N-[2-fluoro-5-(trifluoromethyl)phenyl]urea;
N-[4-Chloro-3-(trifluoromethyl)phenyl]-N-{3-[1-ethyl-4-(1H-pyrrolo[2,3-b]pyridin-4-yl)-1H-pyrazol-3-yl]phenyl}urea;
N-{3-[1-Ethyl-4-(1H-pyrrolo[2,3-b]pyridin-4-yl)-1H-pyrazol-3-yl]phenyl}-N-[3-(methyloxy)phenyl]urea;
N-(2,6-Difluorophenyl)-N-{3-[1-ethyl-4-(1H-pyrrolo[2,3-b]pyridin-4-yl)-1H-pyrazol-3-yl]phenyl}urea;
N-{3-[1-Ethyl-4-(1H-pyrrolo[2,3-b]pyridin-4-yl)-1H-pyrazol-3-yl]phenyl}-N-{4-[(phenylmethyl)oxy]phenyl}urea;
N-Cyclohexyl-N-{3-[1-ethyl-4-(1H-pyrrolo[2,3-b]pyridin-4-yl)-1H-pyrazol-3-yl]phenyl}urea;
N-{3-[1-Ethyl-4-(1H-pyrrolo[2,3-b]pyridin-4-yl)-1H-pyrazol-3-yl]phenyl}-N-(phenylmethyl)urea;
N-{3-[1-Ethyl-4-(1H-pyrrolo[2,3-b]pyridin-4-yl)-1H-pyrazol-3-yl]phenyl}-N-[4-(trifluoromethyl)phenyl]urea;
N-(2-Chlorophenyl)-N-{3-[1-ethyl-4-(1H-pyrrolo[2,3-b]pyridin-4-yl)-1H-pyrazol-3-yl]phenyl}urea;
N-{3-[1-Ethyl-4-(1H-pyrrolo[2,3-b]pyridin-4-yl)-1H-pyrazol-3-yl]phenyl}-N-phenylthiourea;
N-{3-[4-(3-Chloro-1H-pyrrolo[2,3-b]pyridin-4-yl)-1-ethyl-1H-pyrazol-3-yl]phenyl}-N-phenylurea;
N-{3-[4-(3-Bromo-1H-pyrrolo[2,3-b]pyridin-4-yl)-1-ethyl-1H-pyrazol-3-yl]phenyl}-N-phenylurea;
Ethyl 4-(1-ethyl-3-{3-[({[4-(trifluoromethyl)phenyl]amino}carbonyl)amino]phenyl}-1H-pyrazol-4-yl)-1H-pyrrolo[2,3-b]pyridine-2-carboxylate; and
4-(1-Ethyl-3-{3-[({[4-(trifluoromethyl)phenyl]amino}carbonyl)amino]phenyl}-1H-pyrazol-4-yl)-1H-pyrrolo[2,3-b]pyridine-2-carboxylic acid,
and pharmaceutically acceptable salts and solvates thereof.

14. (canceled)

15. A pharmaceutical composition comprising the compound of claim 1, and a pharmaceutically acceptable carrier, diluent or excipient.

16. The pharmaceutical composition according to claim 15 further comprising a chemotherapeutic agent.

17. (canceled)

18. A method for treating a susceptible neoplasm in an mammal in need thereof, said method comprising administering to the mammal a therapeutically effective amount of a compound according to claim 1.

19. A method for treating a susceptible neurotrauma in an mammal in need thereof, said method comprising administering to the mammal a therapeutically effective amount of a compound according to claim 1.

20. The method according to claim 18, wherein said susceptible neoplasm is selected from breast cancer, colon cancer, non-small cell lung cancer, prostate cancer, bladder cancer, ovarian cancer, gastric cancer, pancreatic cancer, carcinoma of the head and neck, esophageal carcinoma, melanoma and renal carcinoma.

21. The method according to claim 19, wherein said susceptible neurotrauma is selected from both open or penetrating head trauma, such as caused by surgery, or a closed head trauma injury, such as caused by an injury to the head region, ischemic stroke, transient ischemic attacks following coronary by-pass, and cognitive decline following other transient ischemic conditions.

22. (canceled)

23. (canceled)

24. (canceled)

25. (canceled)

26. (canceled)

27. (canceled)

28. (canceled)
